(19) United States
(12) Patent Application Publication
(10) Pub. No.: US 2002/0086852 A1
(43) Pub. Date: Jul. 4, 2002

(54) Method for treating respiratory disorders associated with pulmonary elastic fiber injury

(76) Inventors: Jerome O. Cantor, Brooklyn, NY (US); Jing-wen Kuo, Wakefield, MA (US); Paul J. Mihalko, Fremont, CA (US); Dan Sachs, Boston, MA (US); Gerard Turino, New York, NY (US)

Correspondence Address: BRYAN CAVE LLP, 245 Park Avenue, New York, NY, 10167, US

(21) Appl. No.: 09/863,849

(22) Filed: May 23, 2001

Related U.S. Application Data
(63) Continuation-in-part of application No. 09/079,209, filed on May 14, 1998.
(60) Provisional application No. 60/206,612, filed on May 23, 2000.

(51) Int. Cl. A61K 31/737; A61K 31/728; A61K 31/727; A61K 9/00; A61L 9/04

(52) U.S. Cl. 514/54; 514/56; 424/43

(57) ABSTRACT

The present invention relates generally to the field of respiratory therapeutics, and in particular to the treatment of disorders of the lung matrix caused by damage to the elastic fibers of the lung matrix. More specifically, methods and materials are disclosed for the delivery to the lungs of polysaccharides, derivatives thereof and/or drug conjugates, used in the treatment and/or prevention of pulmonary disorders.

### RELATED APPLICATIONS

The present application is a continuation-in-part of pending U.S. patent application Ser. No. 09/079,209, filed on May 14, 1998. The present application also claims the benefit of U.S. Provisional Application No. 60/206,612, filed on May 23, 2000 under 35 U.S.C. § 119(e).

### BACKGROUND OF THE INVENTION

1. Field of the Invention

The present invention relates to the treatment of respiratory disorders caused by either loss of glycosaminoglycans or injury to the pulmonary elastic fiber matrix. More specifically, methods and materials are disclosed for the treatment or prevention of pulmonary disorders such as emphysema, chronic obstructive pulmonary disease (COPD), asthma, cystic fibrosis, inflammatory states, and age-related changes of the lung by delivery to the lungs of polysaccharides or derivatives thereof.

2. Description of the Related Art

Respiratory tract disorders are a widespread problem in the United States and throughout the world. Respiratory tract disorders fall into a number of major categories, including inflammatory conditions, infections, cancer, trauma, embolism, and inherited diseases. Lung damage may also be due to physical trauma and exposure to toxins.

Inflammatory conditions of the respiratory tract include asthma, chronic obstructive pulmonary disease, sarcoidosis, and pulmonary fibrosis. Lung infections include pneumonia (bacterial, viral, fungal, or tuberculin) and viral infections. Cancers in the lung may be primary lung cancer, lymphomas, or metastases from other cancerous organs. Trauma to the lung includes lung contusion, barotrauma, and pneumothorax. Embolisms to the lung can consist of air, bacteria, fungi, and blood clots. Inherited lung diseases include cystic fibrosis, and alpha one antitrypsin deficiency. Toxins that can injure the lung include acidic stomach contents (e.g. aspiration pneumonia), inhaled smoke, and inhaled hot air (e.g. from a fire scene).

Patients with any of the above respiratory tract disorders have a component of lung tissue injury. A common contributor to tissue injury in many of these disorders is related to the influx of inflammatory cells, such as neutrophils, macrophages, and eosinophils. Inflammatory cells release noxious enzymes that can damage tissue and trigger physiologic changes. Elastases are one category of noxious enzyme that inflammatory cells release. Elastase enzymes degrade elastic fibers (elastin) in the lung. The damage caused by elastase enzymes may cause the release of tissue kallikrenin (TK) and may trigger a cascade that attracts additional inflammatory cells to the lung. This influx of additional inflammatory cells release more elastase enzymes, and a “vicious cycle” of lung tissue damage ensues.

Chronic obstructive pulmonary disease (COPD) is a term used to classify two major airflow obstruction disorders: chronic bronchitis and emphysema. Approximately 16 million Americans have COPD, 80% of them were smokers throughout much of their lives. COPD is a leading cause of death in the U.S., accounting for roughly 100,000 deaths per year. Chronic bronchitis is inflammation of the bronchial airways. The bronchial airways connect the trachea with the lungs. When inflamed, the bronchial tubes secrete mucus, causing a chronic cough. Emphysema is an overinflation of the alveoli, or air sacs in the lungs. This condition causes shortness of breath.

In emphysema, the alveolar sacs are overinflated as a result of damage to the elastin skeleton of the lung. Inflammatory cells in emphysematous lungs release elastase enzymes, which degrade or damage elastin fibers within the lung matrix. Emphysema has a number of causes, including smoking, exposure to environmental pollutants, alpha-one antitrypsin deficiency, and aging.

There are no therapies available today to halt the progression of COPD. Inhaled steroids have recently been studied (Lung Health Study II) as a potential therapy to prevent loss of lung function in emphysema patients. The study concluded, however, that inhaled steroids failed to alter the decline in lung function over time. As patients lose lung finction over time, they may become dependent on oxygen, and eventually on ventilators to assist with respiration. A relatively new treatment for patients with emphysema is lung volume reduction surgery. Emphysema patients suffer from air trapping in the lungs. This flattens the diaphragm, impairing the ability to inhale and exhale. Patients with emphysema localized to the upper lung lobes are candidates for lung volume reduction surgery, where the upper lobes are surgically removed to restore the natural concavity and function of the diaphragm.

Acute exacerbations of asthma are often caused by spasm of the airways, or bronchoconstriction, causing symptoms including sudden shortness of breath, wheezing, and cough. Bronchospasm is treated with inhaled bronchodilators (anticholinergics such as ipratropium and beta-agonists such as albuterol). Patients inhale these medications into their lungs as a mist, produced by either a nebulizer or a hand-held meter dose (MDI) or dry powder (DPI) inhaler. Patients with acute episodes may also be treated with oral or intravenous steroids that serve to reduce the inflammatory response that exacerbates the condition.

### SUMMARY OF THE INVENTION

The present invention is directed to a method for the treatment of a variety of respiratory disorders, and more specifically, to the treatment of respiratory disorders associated with elastic fiber injury. The method in accordance with the present invention comprises administration of a polysaccharide or other carbohydrate moiety that binds to elastic fibers. The binding of the polysaccharide to the elastic fibers inhibits enzymes, oxidants, or other injurious agents from contacting and damaging the elastic fibers.

In one mode of the method, the polysaccharide is a glycosaminoglycan. The glycosaminoglycan may be selected from the group consisting of hyaluronic acid, chondroitin sulfate A, chondroitin sulfate B, chondroitin sulfate C, heparan sulfate and heparin. In another mode of the method, the polysaccharide is dextran.

The polysaccharide may be administered to the mammal via a delivery route selected from the group consisting of aerosol inhalation, dry powder inhalation, liquid inhalation and liquid instillation. In one preferred mode, administering the polysaccharide via aerosol inhalation comprises preparing a liquid formulation including the polysaccharide, wherein the concentration of the polysaccharide is less than about 5 mg/ml and the molecular weight of the polysaccharide is less than about 1.5×10⁶ Daltons. The liquid formulation is aerosolized to form a breathable mist such that the particle size of the polysaccharide is less than about 10 microns. A therapeutically effective amount of the polysaccharide is delivered by inhalation of the breathable mist by the mammal.

In a variation to the liquid formulation including the polysaccharide, the molecular weight of the polysaccharide may be less than about 587,000 Daltons. Alternatively, the molecular weight of the polysaccharide may be less than about 220,000 Daltons. In yet another variation, the molecular weight of the polysaccharide may be less than about 150,000 Daltons.

In one preferred mode, the breathable mist is formed by a nebulizer. The nebulizer may operate at a pressure of at least about 15 psi. Alternatively, the nebulizer may operate at a pressure of at least about 30 psi.

In one variation of the present invention, the polysaccharide may be chemically modified. Such modification may include cross-linking, addition of sulfate groups, addition of carboxyl groups, attachment of lipophilic side chains, introduction of acetyl groups, formation of an ester, and/or reaction with a carbodiimide.

Another method in accordance with the present invention, involves administering to a mammal a therapeutic formulation comprising a polysaccharide at a selected dose via a respiratory tract. This method comprises: formulating a solution comprising the polysaccharide to achieve a controlled polysaccharide size of between about 50,000 and 1.5×10⁶ Daltons at a concentration of less than about 5 mg/ml (w/v) of the polysaccharide; producing an aerosol of the solution such that a droplet of the aerosol has a median mass distribution size of between about 0.5-10 microns; and delivering the aerosol into the respiratory tract by inhalation.

The selected dose of polysaccharide is in a range of about 1 μg/kg body weight/day to about 1 mg/kg body weight/day. More preferably, the selected dose is in a range of about 50 μg/kg body weight/day to about 500 μg/kg body weight/day. Still more preferably, the selected dose of polysaccharide is in a range of about 100 μg/kg body weight/day to about 300 μg/kg body weight/day.

In one variation to this method, the solution further comprises a drug. The drug may be selected from the group consisting of terbutaline, albuterol (salbutamol) sulfate, ephedrine sulfate, ephedrine bitartrate, isoetharine hydrochloride, isoetharine mesylate, isoproteranol hydrochloride, isoproteranol sulfate, metaproteranol sulfate, terbutaline sulfate, procaterol, bitolterol mesylate, atropine methyl nitrate, cromolyn sodium, propranalol, fluroisolide, ibuprofin, gentamycin, tobermycin, pentamidine, penicillin, theophylline, bleomycin, etoposide, captopril, n-acetyl cysteine, verapamil, calcitonin, atriopeptin, alpha.-1 antitrypsin (protease inhibitor), interferon, vasopressin, insulin, interleukin-2, superoxide dismutase, tissue plasminogen activator (TPA), plasma factor 8, epidermal growth factor, tumor necrosis factor, heparin, lung surfactant protein, and lipocortin.

In another variation to this method, the polysaccharide is chemically modified. Further to this variation, the solution may further comprise a drug. Preferably, the selected drug exhibits increased solubility or pharmacologic compatibility with the chemically modified polysaccharide, for example, where the polysaccharide is modified to enhance its hydrophobicity. In this mode, the drug may be selected from the group consisting of prostaglandins, amphotericin B, progesterone, isosorbide dinitrate, testosterone, nitroglycerin, estradiol, doxorubicin, beclomethasone and esters thereof, vitamin E, cortisone, dexamethasone and esters thereof, DPPC/DPPG phospholipids, and betamethasone valerete.

In an additional or alternative mode of the present method, the drug may be conjugated to the polysaccharide.

Another aspect of the present invention includes a system for delivering a polysaccharide formulation to a respiratory tract of a mammal. The system comprises: a mixture including a polysaccharide having a molecular weight of between about 50,000 and 1.5×10⁶ Daltons at a concentration of less than about 5.0 mg/ml (w/v) of polysaccharide, and a breathable fluorocarbon propellant; a cannister adapted to contain the mixture under pressure; a valve connected to the cannister for regulating delivery of the mixture; and a nozzle interconnected with the valve for converting the pressurized mixture inside the cannister into an inhalable aerosol mist when the valve is actuated, and the mixture is via the nozzle outside the cannister.

In one embodiment of the system, the polysaccaride in the aerosol mist has a median mass distribution size of between about 0.5-10 microns. In a variation to the system, the mixture may also comprise a drug.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the preferred embodiments which follow.

### BRIEF DESCRIPTION OF THE DRAWINGS

FIG. 1. HA exerts a protective effect on air-space enlargement when given at different times relative to pancreatic elastase.

FIG. 2. HA exerts a protective effect on air-space enlargement when given 2 hrs prior to human neutrophil elastase.

FIG. 3. Incubation of hyaluronic acid with elastase increases, rather than reduces, degradation of elastin, as measured by release of radioactivity from ³H-elastin substrate. Thus, HA has no elastase inhibitory capacity.

FIG. 4. Chromatographic separation of bovine tracheal HA on Sephacryl S-500 gel column.

FIG. 5. High-power view of fluorescent elastic fibers in alveolar septa (arrowheads), 1 hr after instillment of fluorescein-labeled HA. (Original magnification: ×790)

FIG. 6. Elastic fibers in a large pulmonary blood vessel show prominent fluorescence, 2 hrs after instillment of fluorescein-labeled HA (Original magnification: ×250)

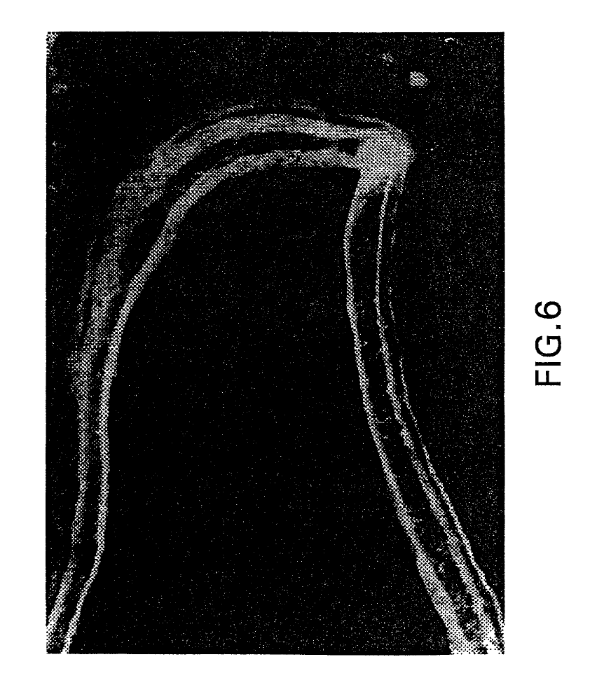

FIG. 7. The effect of aerosolized HA on the percentage of neutrophils in lung lavage fluid at 24 hrs (N=3 for all groups; T bars indicate SEM)

FIG. 8. (Upper Left) Cultured rat pleural mesothelial cells showing characteristic polygonal shape; (Upper Right) Phase contrast photomicrograph demonstrating prominent extracellular matrix, which appears black; (Lower Left) Fluorescence photomicrograph of cell-free rat pleural mesothelial matrix following incubation with fluorescein-labeled HA (1 mg/ml) for 10 min. Note preferential binding of fluorescein-HA to extracellular matrix; (Lower Right) Following exposure of cell free matrix to porcine pancreatic elastase (100 ng/ml) for 1 hr, much of the fluorescein-HA is removed. However, residual fluorescence indicates that the matrix remains largely intact. The elastase-induced loss of fluorescence suggests that HA preferentially binds to elastic fibers.

FIG. 9. Although pretreatment of the cell-free matrices with 1 mg/ml HA reduced the amount of radioactivity released by either 1 ig/ml or 100 ng/ml porcine pancreatic elastase, the protective effect was much more pronounced with the lower concentration of the enzyme (p<0.001). T bars indicate SEM.

FIG. 10. Fluorescence photomicrograph showing binding of a second preparation of HA to rat pleural mesothelial cell elastic fibers. This shows that the protective effect of HA is not limited to a specific preparation of the material.

FIG. 11. Shows the protective effects of various GAGs on elastic fiber matrix in vitro.

FIG. 12. Shows the protective effects of various polysaccharides on elastic fiber matrix in vitro.

FIG. 13. Shows a comparison of the protective effects of three different Chondroitin Sulfates and the 3 different molecular weight HA specimens against controls in vitro.

FIG. 14. Illustrates the protective effects of two different molecular weight HA formulations compared with two different concentrations of PPE in vitro.

FIG. 15. Shows that the typical nebulizer droplet size distribution tends to be bimodal.

### DETAILED DESCRIPTION OF THE PREFERRED EMBODIMENT

Elastic fibers are a prominent component of the extracellular matrix and play an important role in determining the mechanical properties of tissues. By virtue of their distensibility, elastic fibers permit tissues to function normally despite the application of external forces. In the lung, for example, interstitial and pleural elastic fibers facilitate tissue recoil following inspiration, preventing permanent distention of the organ and maintaining the flow of gases within airways. Damage to these fibers causes dilatation and rupture of alveoli, resulting in pulmonary emphysema (Janoff et al. (1985) Am. Rev. Respir. Dis. 132:417-433; Senior and Kuhn (1988), In Fishman (ed), Pulmonary Diseases and Disorders, 2d ed. New York, McGraw-Hill, p. 1209-1218).

Despite the importance of maintaining the integrity of elastic fibers, there is currently no effective means of protecting them from damage. Since these fibers are susceptible to degradation by elastases, as discussed above, various elastase inhibitors have been tested as a possible means of preventing elastic fiber injury (Janoff et al. (1985) Am. Rev. Respir. Dis. 132:417-433; Zimmerman and Powers (1989), In Hornebeck (ed), Elastin and Elastases, vol II, Boca Raton, CRC Press, p. 109-123). In particular, a naturally occurring inhibitor, alpha-1-antiproteinase, has been given to individuals who normally lack this inhibitor in an attempt to slow the progression of elastic fiber breakdown which leads to pulmonary emphysema (Laurell and Eriksson (1963) Scand. J. Clin. Lab. Invest. 15:132-140). Such a treatment strategy assumes, however, that elastic fiber injury is caused by a specific type of biochemical derangement, i.e. alpha-1-antiproteinase deficiency. If damage to these fibers represents a more general reaction to a variety of insults (with elastases playing a variable role), then enzyme inhibition may have only limited efficacy. The subject invention is directed to inhibition of pulmonary tissue elastic fiber injury by administration of polysaccharides or carbohydrate moieties that bind to and coat elastic fibers, thereby inhibiting enzymes, oxidants, or other injurious agents from damaging these fibers.

The present invention discloses methods and materials for the treatment or mitigation of pulmonary disorders by delivery to the lungs of polysaccharides and/or derivatives thereof. The polysaccharide formulations disclosed herein may be useful in treating and/or preventing a variety of pulmonary conditions and disorders, including for example emphysema, as detailed in U.S. Pat. No. 5,633,003 to Cantor and co-pending U.S. patent application Ser. No. 09/079,209; the disclosures of which are incorporated herein in their entirety by reference thereto for all purposes. In addition, other therapeutic indications for polysaccharide administration to the lung includes: stabilizing the lung matrix (tissue which contains the alveolar sacs and bronchii) by forming a polymer network within the lung matrix; placing a polysaccharide barrier on the matrix fibers of the lung to reduce or eliminate future degradation of the lung fibers, or to protect the fibers from noxious agents while they undergo repair; providing a polysaccharide coating of the lung matrix, surface, bronchioles, and/or alveoli that enhances the moisture content, lubrication, or elastic recoil of the lung; replacing HA in conditions where HA is diminished (e.g. aging, emphysema); providing a bulking agent in the lung to reinforce delicate anatomic structures such as alveolar walls (e.g. blebs); providing a lubricant between the internal & external pleura; providing a viscoelastic agent to facilitate elastic lung recoil; providing a dressing to facilitate healing of injured lung tissue; reducing and/or preventing inflammation due to infection, cancer, irritation, allergy, etc.; treating bronchospasm; lubricating and/or loosening mucous; binding to cell receptors to influence cell activity in the lung, such as ciliary cell beating, cell attachment (or adhesion), or cell migration.

The concept that pulmonary emphysema is caused by an imbalance between proteinases and their inhibitors has served to focus research on the role of elastases with the hope that inhibiting the activity of these enzymes will prevent lung injury. Such a treatment strategy assumes, however, that emphysema is caused by a single abnormality; namely, excess elastase activity. If the disease represents a more general response of the lung to a variety of insults (with elastases playing a variable role), then enzyme inhibition may have only limited efficacy and other forms of treatment may be required.

An alternative approach to alveolar destruction may involve the use of polysaccharides to directly protect lung elastic fibers from injury. Polysaccharides preferentially bind to elastic fibers, prevent elastolysis and limit air-space enlargement in experimental models of emphysema induced by either pancreatic or neutrophil elastase. Since elastic fiber breakdown may be a final common pathway in the disease process, this form of treatment will be effective against a number of agents capable of causing emphysema, including various oxidants present in air pollutants and cigarette smoke.

Pretreatment of the lung with hyaluronidase to reduce its HA content results in an additional increase in air-space enlargement over that induced by intratracheal instillation of elastase in lungs with normal HA content. HA is also significantly reduced in the lungs of patients with pulmonary emphysema. Without being limited to any mechanism, it is believed that locally high concentrations of HA may act to reduce contact between a neutrophil or macrophage in contact with elastic fibers. By this mechanism, HA could act to prevent direct cell-mediated elastic fiber damage. The mechanism may also involve formation of electrostatic or hydrogen bonds between these two components. Such binding sites may not be situated on the elastin protein itself, but may instead involve surrounding structures.

HA may also protect elastic fibers by virtue of its ability to retain water. Loss of HA can decrease extravascular water content in the lung interstitium. Negatively charged carboxyl groups attached to the saccharide moieties of HA repel one another, enlarging the domain of HA and enhancing its ability to entrap water. This process may cause an increase in viscosity that reduces the movement of surrounding molecules, including elastases, thereby limiting injury to elastic fibers.

Oxidants include oxidants involved in tissue and/or elastic fiber injury which include but are not limited to, ozone, superoxide anion, hydrogen peroxide, hydroxyl radical, hypochlorous acid, monochloramine, nitrogen dioxide, and peroxyl radical.

Other injurious agents include ultraviolet radiation, infectious agents, genetic abnormalities, aging and toxic substances, (e.g. insecticides, exhaust fumes, and chemotherapeutic agents). Genetic abnormalities include alpha-1-antiproteinase deficiency and other types which impair elastic fiber synthesis or promote elastic fiber degradation.

Binding in the context of the present invention includes both covalent and non-covalent binding. The binding may be either high or low affinity. The binding may be temporary such that the binding is a coating sufficient to provide a temporary interation. Examples of binding forces include, but are not limited to, ionic and covalent bonds, hydrogen binding, electrostatic forces, dipole interactions, or Van der Waals forces. Binding can be defined empirically by those skilled in the art by fluorescence microscopy, following conjugation of the compound with a fluorescent dye, as discussed in greater detail below.

The treatment is intended for a variety of mammals including humans.

The polysaccharide or carbohydrate moiety may be administered alone or in combination with other polysaccharides or carbohydrate moieties, with or without a suitable carrier. Such suitable carriers include, but are not limited to, carriers like saline solution, DMSO, alcohol, or water. It may be composed of naturally occurring, chemically modified, or artificially synthesized compounds which are wholly or partially composed of polysaccharides or other carbohydrate moieties, and which are capable of binding to elastic fibers.

The amount of the polysaccharide or carbohydrate moiety administered daily may vary from about 1 ig/kg to about 1 mg/kg of body weight, depending on the site and route of administration. More preferably, the dose is in a range of from about 50 μg/kg body weight/day to about 500 μg/kg body weight/day. Most preferably, the dose is in a range of from about 100 μg/kg body weight/day to about 300 μg/kg body weight/day. For example, a 50 minute exposure to an aerosol containing a 0.1% solution of bovine tracheal hyaluronic acid (HA) in water (1 mg/ml) was effective in coating hamster lung elastic fibers with HA.

In one aspect of the present invention, a method for using a formulation comprising a polysaccharide to treat and/or prevent a respiratory disorder. In one aspect, the method comprises the steps of selecting formulation parameters, which include the molecular weight, the concentration and the viscosity of polysaccharide, such that when aerosolized, the formulation yields a droplet size adapted for delivery to the lungs. The formulation is then aerosolized to form an aerosol, and delivered to the lungs.

Another aspect of the invention relates to a method for delivering to the lung alveoli, also referred to as the respiratory zone or deep lung, a polysaccharide or derivative thereof. The method comprises selecting a preparation of the polysaccharide or derivative having a molecular weight sufficient to provide a desired therapeutic profile. Then, preparing a delivery formulation comprising the selected preparation of polysaccharide or derivative at a concentration which when aerosolized yields a particle size suitable for delivery to the deep lung. The delivery formulation is then aerosolized to form an aerosol, and delivered to the deep lung.

In another mode of the method for delivering to a lung alveolus an amount of a formulation comprising a polysaccharide or derivative, formulation parameters are selected. These parameters include molecular weight, concentration and viscosity of the polysaccharide or derivative, such that when aerosolized, the formulation yields a droplet size adapted for delivery to the lung alveoli.

Another aspect of the invention relates to a method of treating and/or preventing respiratory disorders by the use of hyaluronic acid, its derivatives, other polysaccharides, and other polysaccharides, either alone or in conjunction with pharmaceuticals, delivered by nebulization or instillation, etc., to the lung tissues.

Another aspect of the invention relates to a method for delivering to a selected target site in a lung, a polysaccharide or derivative thereof. The method comprises the steps of preparing a formulation comprising the polysaccharide or derivative at a molecular weight and concentration adapted to yield a desired rheological profile for effective mass transfer during aerosolization or nebulization; and selecting a delivery apparatus and operation parameters, such that when aerosolized, the formulation yields a median droplet size of less than 10 microns, preferably less than 5 microns and most preferably between 0.05-5 microns, with the size range of approximately 2-5 microns being adapted for delivery to conducting airways, or the size range of approximately 0.5-2 microns being adapted for delivery to the deep lung or respiratory zone.

Another aspect of the invention relates to a formulation comprising HA, other polysaccharides and derivatives thereof having a molecular weight, a concentration and a viscosity that are selected to provide a desired therapeutic profile, and to be deliverable by aerosolization to the deep lung for the treatment of a respiratory disorder.

Another aspect of the invention relates to a formulation comprising HA conjugated with a second active agent, wherein the formulation has a molecular weight, a concentration and a viscosity that are selected to be deliverable in aerosol form to an alveolus for the treatment of a respiratory disorder.

Another aspect of the invention relates to a formulation comprising a polysaccharide and a second agent, wherein the formulation is adapted to be delivered to a lung and also adapted to provide systemic delivery of the second agent.

The purpose of the present invention is to provide means to deliver bio-compatible polymers and/or derivatives thereof, for the treatment or mitigation of pulmonary disorders. The polysaccharide formulations disclosed herein may be useful in treating and/or preventing a variety of pulmonary conditions and disorders, including for example emphysema, as detailed by Cantor in U.S. Pat. No. 5,633,003; the disclosure of which is incorporated herein in its entirety by reference thereto. In addition, other therapeutic indications for polysaccharide administration to the lung includes: stabilizing the lung matrix (tissue which contains the alveolar sacs and bronchii) by forming a polymer network within the lung matrix; placing a polysaccharide barrier on the matrix fibers of the lung to reduce or eliminate future degradation of the lung fibers, or to protect the fibers from noxious agents while they undergo repair; providing a polysaccharide coating of the lung matrix, surface, bronchioles, and/or alveoli that enhances the moisture content, lubrication, or elastic recoil of the lung; replacing hyaluronic acid (HA) in conditions where HA is diminished (e.g. aging, emphysema); providing a bulking agent in the lung to reinforce delicate anatomic structures such as alveolar walls (e.g. blebs); providing a lubricant between the internal & external pleura; providing a viscoelastic agent to facilitate elastic lung recoil; providing a dressing to facilitate healing of injured lung tissue; reducing and/or preventing inflammation due to infection, cancer, irritation, allergy, etc.; treating bronchospasm; lubricating and/or loosening mucous; binding to cell receptors to influence cell activity in the lung, such as ciliary cell beating, cell attachment (or adhesion), or cell migration.

The biocompatible polymers useful in the present invention include without limitation, natural and synthetic, native and modified, anionic or acidic saccharides, disaccharides, oligosaccharides, polysaccharides and in particular, the glycosaminoglycans (GAGs) or acid mucopolysaccharides, which include both non-sulfated (e.g., HA and chondroitin) and sulfated forms (e.g., chondroitin sulfate, dermatan sulfate, heparan sulfate, heparin sulfate, and keratan sulfate). This class of acid mucopolysaccharides can be defined more generally as any polysaccharide having a repeating unit of a dissacharide composed of a hexosamine, e.g., N-acetylated glucosamine, and a uronic acid, e.g., D-glucuronic acid, with or without a sulfate group. Also included within the class of polysaccharides in accordance with the present invention are dextrans, lectins, glucans, mannans, polyethylene glycol (PEG), as well as polypeptides and proteins. In one variation of the present invention, the formulation may comprise a combination of one or more polysaccharides. In addition, this invention is intended to cover polymer derivatives that may be produced by the addition of various chemical groups, such as hydroxyl, carboxyl, sulfate groups, or bonded to the polymer.

In accordance with one aspect of the invention, polysaccharides may be obtained via any variety of methods in the prior art such as bacterial fermentation, via processing from animal or plant tissue, or via chemical synthesis. The formulation of the material will enable delivery of the polysaccharides into the lung via aerosol, dry powder delivery, or direct instillation in such a fashion as to adequately cover target, or susceptible, or diseased tissue. Specifically, the concentration, molecular weight, and viscosity will be such that the material can be dispersed throughout the target site(s) within the lungs, and allow for a desired dosing frequency (e.g., preferably about every six hours to once per day). The material is preferably free from impurities or bacteria that may render it unsafe for human use.

HA is one of the GAGs naturally present in the matrix of human lung. It plays a number of roles, including acting as a lubricant, and interacting with various cells and molecules in the lung environment. It is secreted by mesothelial cells in response to congestive heart failure, acute respiratory distress syndrome (ARDS), and other respiratory tract abnormalities. As used herein, the term HA means hyaluronic acid and any of its hyaluronate salts, including, for example, sodium hyaluronate (the sodium salt), potassium hyaluronate, magnesium hyaluronate, and calcium hyaluronate.

HA is a polymer consisting of simple, repeating disaccharide units. These repeating disaccharide units consist of glucuronic acid and N-acetyl glycosamine. It is made by connective tissue cells of all animals, and is present in large amounts in such tissues as the vitreous humor of the eye, the synovial fluids of joints, and the roostercomb of chickens. One method of isolating HA is to process tissue such as roostercombs. This invention can utilize HA isolated and purified from natural sources, as described in the prior art; HA isolated from natural sources can be obtained from commercial suppliers, such as Biomatrix, Anika Therapeutics, ICN, and Pharmacia.

Another method of producing HA is via fermentation of bacteria, such as streptococci. The bacteria are incubated in a sugar rich broth, and excrete HA into the broth. HA is then isolated from the broth and impurities are removed. The molecular weight of HA produced via fermentation may be altered by the sugars placed in the fermentation broth. This invention can utilize HA produced by bacterial fermentation as described in the prior art; HA produced via fermentation can be obtained from companies such as Bayer, Genzyme, and Lifecore Biomedical.

In its natural form, HA has a molecular weight in the range of 5×10⁴ up to 1×10⁷ Daltons. Its molecular weight may be reduced via a number of cutting processes such as exposure to acid, heat (e.g. autoclave, microwave, dry heat), or ultrasonic waves. HA is soluble in water and can form highly viscous aqueous solutions.

HA obtained from either animal tissue (e.g. roostercombs) or bacterial fermentation may contain contaminant proteins. Inhalation of protein contaminants may induce an allergic reaction in certain patients, causing bronchoconstriction, edema, and influx of inflammatory cells to the lung. Therefore, the HA of the invention have a protein content of less than 5%, more preferably less than 2%, and most preferably from 0% to undetectable levels. HA preparations may also contain endotoxin contaminants. To minimize the risk of an allergic reaction, the HA of the invention have an endotoxin concentration of less than 0.07 EU/mg, and preferably less than 0.01 /EU/mg, and most preferably from 0% to undetectably levels.

The polysaccharides may serve as medium for bacterial growth. To insure that delivery of polysaccharides to the lung does not induce pneumonia, the material should be sterile. Thus, the polysaccharides of the invention have a bacterial count of less than 1 cfu/g, preferably zero.

Other physiologic parameters of the polysaccharides for use in the lung include pH between 4.0 to 8.9, and nontoxic concentrations of heavy metals, as judged by the criteria established for USP water for inhalation.

In one mode of the invention, a liquid formulation of polysaccharides is used. The liquid may be aerosolized for inhalation as a mist via an aerosolization device such as a nebulizer, atomizer, or inhaler.

In accordance with another mode, the formulation is a dry powder which individuals would mix at home or the hospital with saline or water before instillation to an aerosol device. The device would produce an aerosol for inhalation by the patient. A dry powder formulation could also be delivered in powder form by an aerosol device, such as air gun powered aerosol chamber. Companies which produce dry powder delivery devices include Dura Delivery Systems (the “Dryhaler”), Inhale Therapeutics, and Glaxo Wellcome (Diskhaler).

The respiratory system consists generally of three components: the tracheal/pharyngeal, the bronchial and the alveolar. It is known that particles of 10-50 microns migrate to the tracheal/pharyngeal component. Particles of about 5-10 microns migrate to the bronchial component, and particles of 0.5 to 5 microns migrate to the alveolar component. Particles less than 0.5 microns in size are not retained.

The mass median aerodynamic diameter (MMAD) is predictive of where in the lung a given particle will end up. The MMAD is usually expressed in microns. A related parameter is the geometric standard deviation (GSD). A GSD of 1 is equal to a normal distribution. A GSD of less than one indicates a narrow size dispersion and a GSD of more than 1 indicates a broad size dispersion.

Chemical modifications of polysaccharides may be used to produce new compounds which can bind to lung elastic fibers with an increased affinity. Elastin is a cationic protein. Consequently, introducing negatively charged groups, ions or substitutions can enhance the electostatic forces between the polysaccharide and the elastic fibers. For example, sulfate groups could be added to make the compound more negatively charged.

Various specific chemical modification schemes for HA are provided below. One skilled in the art could readily adapt these schemes to modify other polysaccharides.

Sulfate can be introduced to HA's hydroxyl groups, especially the 6-hydroxyl of the N-acetylglucosamine moiety, by the following reactions:

1. Reaction of tetrabutylammonium salt of HA with SO₃ -pyridine as detailed in U.S. Pat. No. 6,027,741, entitled “Sulfated hyaluronic acid and esters thereof”; incorporated herein in its entirety by reference thereto.

2. Reaction of dry HA with chlorosulfonic acid in dry pyridine, as described by Wolfrom, M L, “Chondroitin sulfate modifications” J. Am. Chem. Soc. 82, 2588-2592.

Another means of adding sulfate groups to HA involves reaction with NH₂ after deacetylation of N-acetyl. The sulfation is completed in two steps, (a) deacetylation of N-acetylglucosamine moeity of HA by its reaction with anhydrous hydrazine at elevated temperature, followed by (b) treatment of the derived product with trimethylamine-sulfur trioxide. See e.g., U.S. Pat. No. 5,008,253, entitled “Sulfoamino derivatives of chondroitin sulfates of dermatan sulfate and of hyaluronic acid and their pharmacological properties”; the disclosure of which is incorporated herein in its entirety by reference thereto.

In addition to sulfate groups, carboxyl groups can be added to polysaccharides to increase their negative charge, thereby improving their binding to elastin in the lung matrix. The following reactions are provided to illustrate carboxylation schemes reactions for HA:

1. The 6-hydroxyl of the N-acetylglucosamine can be a target for further modification to introduce an additional carboxyl group, for example, reaction of dry HA with sodium chloroacetate.

2. The hydroxyl functional groups of HA are esterified by converting the carboxyl functional groups of HA into a tertiary ammonium or tertiary phosphonium salt in the presence of water and aprotic solvent and then treating the solution with succinic anhydride, as disclosed in U.S. Pat. No. 6,017,901, entitled “Heavy metal salts of succinic acid hemiesters with hyaluronic acid or hyaluronic acid esters, a process for their preparation and relative pharmaceutical compositions.

3. Similar to the previous example, dianhydrides such as ethylenediamine tetraacetic acid dianhydride (EDTAA) can be used. This reaction produces crosslinked HA. However, free pendant carboxyl groups from the anhydride may exist after the reaction of dianhydrides and HA, as described in U.S. Pat. No. 5,690,961, entitled “Acidic polysaccharides crosslinked with polycarboxylic acids and their uses”. Each of the above references are incorporated in their entirety by reference thereto.

Lipophilic side chains can also be attached to polysaccharides to increase the binding strength between the polysaccharide and elastin. Polar functional groups such as carboxyl and hydroxyl groups impart hydrophilicity. The introduction of lipophilic moieties to the polysaccharide can improve their affinity for elastin fibers, because elastin has a composition that is rich in amino acids with aliphatic side chains. The following reaction schemes are provided with respect to HA:

1. The introduction of an acetyl group to HA at its four hydroxyl site produces acetylhyaluronate. A method of manufacturing acetylhyaluronate comprises the steps of suspending hyaluronic acid powder in an acetic anhydride solvent and then adding concentrated sulfuric acid thereto to effect acetylation. The maximum degree of substitution is four, since there are four hydroxyl groups in each dissacharide unit of HA. Practically, only partial acetylation occurs. The degree of substitution determines the lipophilicity (thus hydrophobicity) of the modified HA. The more lipophilic, the higher the affinity of HA derivatives to the lipophilic moiety of elastin fibers. See e.g., U.S. Pat. No. 5,679,657, entitled “Low molecular weight acetylhyaluronate, skin-softening composition, method of manufacturing the same, and method of purifying the same”.

2. HA can react with alkylhalide, such as propyl iodide to form the ester function from the carboxyl group. The HA derivatives are less water-soluble and more lipophilic, proportional to the increase of degree of derivatization, as described in European Patent Application No. 86305233.8.

3. The reactions of free hyaluronic acid and diazomethane produce the methyl ester of HA, as described by Jeanloz et al., J. Biol. Chem. 186 (1950), 495-511.

4. Carbodiimides with aliphatic or aromatic side chains react with the carboxyl group of hyaluronic acid to form acylurea derivatives of HA with hydrophobic features, as described by Kuo et.al, Bioconjugate Chemistry, 1991,2, 232-241. Each of the above references is incorporated herein in their entirety by reference thereto.

In a preferred aspect of the present invention, a molecular weight of the polysaccharide or derivative is selected to produce a desired physiologic effect or molecular interaction, i.e., a desired therapeutic profile. As discussed above, the polysaccharides and their derivatives are polymers of repeating units and as a result, may be isolated, purified, synthesized, and/or commercially obtained in a wide range of molecular weights. The physiologic effects and molecular interactions of the polymers vary with molecular weight. Likewise, the physical delivery of the polymers to a selected target site within the lung also varies with polymer size (molecular weight). Different therapeutic profiles would be desirable for different clinical indications, and can be individually developed and optimized without undue experimentation by a physician skilled in the art, using the teachings disclosed herein.

For example, where protection of extracellular matrix against damage is desired, a high molecular weight preparation of polysaccharide would be desirable in order to provide effective binding to and coating of elastin fibers. Indeed, a high molecular weight polysaccharide derivative, modified to enhance its affinity for elastin, would be preferred. High molecular weight preparations are also preferred for depot of drugs, where the large polymer may be a better excipient, a better carrier and better for addressing large airway diseases. Alternatively, lower molecular weight preparations may be better for loosening sputum, penetrating to the deep lung tissues, and traversing alveolar-epithelial barrier. In addition, where high water tension and excessive resistance to alveolar expansion is present (e.g., respiratory distress syndrome), lower molecular weight preparations of the polymers, which are less hydroscopic and provide less inherent elastic recoil, may be preferred. In selecting the molecular weight, the physician will have to balance the desired therapeutic profile against physical restraints on delivery into the deep lungs.

A “therapeutic profile” as used herein comprises at least one of the following indices: duration (e.g., half-life) of the glycosaminoglycan in the lung, ability to retain water, elastic recoil, coating index, binding affinity for elastin (or other extracellular matrix components known in the art), absorption into systemic circulation, etc.

It has been observed that higher molecular weight preparations of polysaccharides: (1) persist longer in the lungs, (2) hold more water, (3) provide greater supplementation of elastic recoil, and (4) provide thicker and more complete coverage of extracellular matrix, than lower molecular weight preparations.

With respect to duration in the lungs, a polymer preparation in accordance with the present invention may have a molecular weight that resides in the lung for between 0.5 hour and one week, preferably between 1 hour and one day, and more preferably between 4 and 16 hours. Most preferably, a GAG will remain associated with the lung matrix for at least 6 hours. This would allow for dosing four or less time a day.

It has been observed that molecular weights of HA preparations for between 25,000 Daltons and 2,000,000 Daltons can be used to provide lung duration times, water retention, elastic recoil, and matrix coverage, consistent with the above. The relationship between polysaccharide concentration, molecular weight and viscosity is discussed in greater detail below. When a preparation of HA having a molecular weight of greater than 2,000,000 Daltons was used, it produced a solution that was excessively viscous. Thus, although the highest molecular weight preparations yield the greatest duration times, water retention, elastic recoil and matrix coverage, these properties must be balanced against excessive viscosity, particularly at lower deployment temperatures (e.g., jet nebulizers that cool the solutions significantly during expansion). In general, it has been observed for HA, that it was preferred to use a preparation having a molecular weight of less than about 1.5×10⁶ Daltons, more preferably less than 500 kD, more preferably still, less than about 220 kD, and most preferably less than about 150 kD.

Besides the molecular weight, the concentration of the glycosaminoglycan solution also influences duration times, water retention, elastic recoil, and matrix coverage, and formulation viscosity. Viscosity increases with increasing concentration. Viscosity increases with decreasing temperature. Concentrations of HA are preferably between about 0.05 mg/L and 5 mg/L at ambient temperature (20° to 25° C.). The preferred concentration is less than 5 mg/L, more preferably less than 2 mg/L, and more preferably less than 1 mg/L. The preferred concentration is above 0.05 mg/L, more preferably over 0.5 mg/L. The concentration of a selected molecular weight preparation may be adjusted to yield a selected viscosity, depending on the temperature.

The viscosity or thickness of the material is related to the combination of concentration and molecular weight. Viscosity increases with increasing molecular weight if concentration remains constant. Likewise, viscosity increases with increasing concentration if molecular weight remains constant. Viscosity can be measured by a viscometer (one such device is manufactured by the company Brookfield), and is expressed in units of centipoises (abbreviation: cps).

The material must be transferred from the delivery device (e.g. via an aerosolization device) into the respiratory tract, down to the distal bronchi and alveoli, from where it can diffuse into the extracellular lung matrix. The delivery formulation should have physical characteristics which avoid clogging of the aerosol device and clumping of aerosolized particles. It should be noted that a viscous material, delivered slowly, may not cause clogging or plugging, whereas a less viscous material may, if delivered quickly.

Formulations of specific molecular weight, concentration and viscosity are preferably produced by adding a volume of sterile delivery solvent (e.g., water or saline) to an amount of sterile, medical grade polysaccharide powder. More preferably, unit dose vials containing a pre-weighed dose of polysaccharide may be dissolved just prior to use by injection of sterile solvent into the sealed vial. The powdered polysaccharide is then mixed in the solvent until dissolved. Alternatively, polysaccharide of a certain concentration can be prepared by diluting liquid polysaccharide with sterile solvent.

Formulation temperatures of between about 0° to about 100° C., preferably between about 4° and 60° C. and more preferably between about 15° and 37° C. may be used in accordance with the present invention; however, the viscosity of a given molecular weight and concentration of a polysaccharide varies with temperature. Thus, the user can determine empirically the viscosity with a viscometer, and adjust the concentration accordingly to yield a viscosity adapted for delivery by the desired delivery mechanism (e.g., nebulizer, aerosolizer, inhaler etc.) to the selected target site in the lungs. It has been observed that for delivery to the lungs at ambient temperature, the viscosity is preferably below about 1000 cps, more preferably below about 100 cps, and most preferably below about 50 cps.

Another factor which should be considered in formulating a polysaccharide solution for delivery to a selected target site in the respiratory tract is the droplet or particle size generated. This factor should be considered for aerosol as well as powder delivery pathways. Particle size is preferably below about 10 microns in diameter. More preferably, the particle size is between 2 and 5 microns. The relationship between particle size in microns and fluorescence-labeled polysaccharide molecular weight and concentration can be measured as the Mass Median Aerodynamic Diameter using a Cascade Impactor (see data in Examples below). The numbers on the x-axis represent sieve sizes in microns and the numbers on the y-axis represent fluorescence (i.e., amount of polysaccharide) which impacts on the particular sieve (i.e., median particle size is too large to fit through the pores). A humidified variation of the Cascade Impactor can also be used to more closely reflect pulmonary delivery, because the polymers of the present invention may be hydroscopic and therefore absorb water and swell in size.

Raabe et al., reported a survey of particle size access to various airways in small laboratory animals using inhaled monodisperse aerosol particles. Raabe et al., Ann. Occup. Hyg. 1988, 32:53-63; incorporated herein by reference thereto. Similar analysis may be performed to inform the clinician as to the desirable particle size for delivery to a target site within the lung.

Particle size in accordance with a preferred mode of the present invention may be between about 2 microns and about 5 microns, thereby being adapted for delivery into the lung alveoli. Larger size particles are not as efficiently delivered through the distal bronchioles, whereas much smaller sizes tend to be exhaled before contacting the alveolar lining. Thus, whereas the therapeutic profile (e.g., duration, water retention, elastic recoil and matrix coverage) tend to increase with increasing molecular weight, the relative deliverability (i.e., frequency of particles within the 2-5 micron range) tends to decrease with increasing molecular weight.

In order to produce an aerosol which can be inhaled by human beings for distribution throughout the lung, the glycosaminoglycan must be aerosolized into appropriate droplet sizes as detailed above, preferably between about 2-5 microns in diameter. Some droplets larger than 5 microns in diameter may deposit in the nebulizer tubing or mask, mouth, pharynx, or laryngeal region. Droplets less than 2 microns in diameter tend not to be deposited in the respiratory tract, but are exhaled and lost. Droplet sizes of 2-5 microns can be achieved by selection of appropriate aerosol devices, solution concentration, compound molecular weight, and additives, in accordance with the teachings herein.

Additives such as surfactants, soaps, Vitamin E, and alcohol may be added to avoid clumping of droplets after they are produced, and to facilitate generation of small particles from an aerosol device. One embodiment of the invention includes glycosaminoglycans in combination with one or more of these additives.

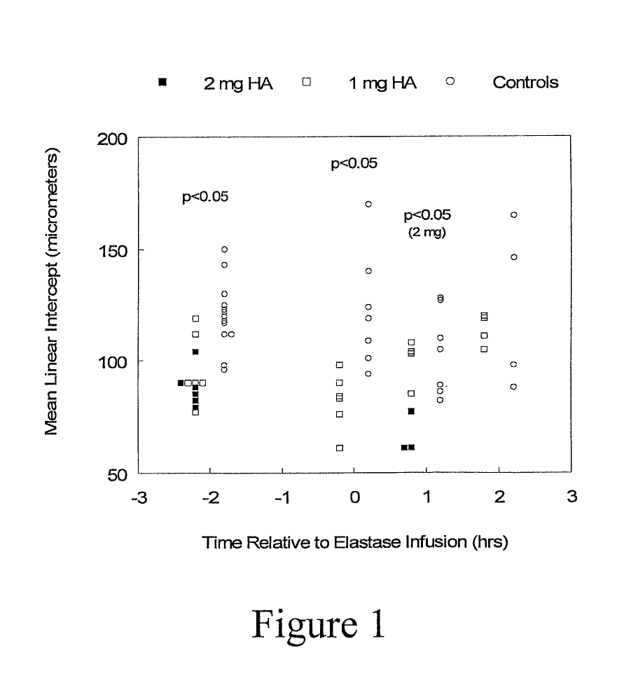

A method of selecting breathable formulations for delivery to the lung by aerosol is to screen multiple formulations for those formulations which will produce droplets of less than 10 microns in diameter, more preferable less than 6 microns, most preferably 2-5 microns. Formulations which produce droplets larger than 10 microns are not suitable for delivery into the lung. Particle size distribution of the aerosolized mist for each formulation is measured with a device such as a Malvern Laser or a Cascade Impactor (as used to generate the data shown in FIGS. 1A-L). This invention includes all molecular weight and concentration combinations of polysaccharides that can be aerosolized into droplet sizes of under 10 microns, and more preferably between about 2-5 microns.

One embodiment of the invention involves use of an aerosol-generating device to produce an inhalable mist. One class of device to generate polysaccharide aerosols is a spray atomizer. Another class of device to generate polysaccharide aerosols is a nebulizer. Nebulizers are designed to produce droplets under 10 microns.

Many commonly used nebulizers may be used to aerosolize polysaccharides for delivery to the lung: 1) compressed air nebulizers (examples of these include the AeroEclipse, Pari L. C., the Parijet and the Whisper Jet) and 2) ultrasonic nebulizers. Compressed air nebulizers generate droplets by shattering a liquid stream with fast moving air. One mode of the invention involves use of a compressed air nebulizer to aerosolize polysaccharide solutions into droplets under 10 microns in size. Ultrasonic nebulizers use a piezoelectric transducer to transform electrical current into mechanical oscillations, which produces aerosol droplets from a liquid solution. Droplets produced by ultrasonic nebulizers are carried off by a flow of air. Another mode of the invention involves the use of an ultrasonic nebulizer to aerosolize polysaccharide solutions into droplets less than 10 microns in size.

Another mode of this invention is use of a hand-held inhaler to generate polysaccharide aerosols. This portable device will permit an individual to administer a single dose of mist, rather than a continuous “cloud” of mist into the patient's mouth. Individuals with bronchoconstrictive diseases such as asthma, allergies, or COPD often carry these hand-held inhalers (e.g., MDI and DPI) in their pocket or purse for use to alleviate a sudden attack of shortness of breath. These devices contain bronchodilator medication such as albuterol or atrovent. They would also be a convenient way to deliver glycosaminoglycan to patients.

For treatment via nebulizer, patients would inhale the aerosolized polysaccharide solution via continuous nebulization, similar to the way patients with acute attacks of asthma or emphysema are treated with aerosolized bronchodilators. The aerosol may be delivered through tubing or a mask to the patient's mouth for inhalation into the lungs. Treatment time may last 30 minutes or less. The mouth is preferably used for inhalation (rather than the nose) to avoid “wasted” nasal deposition. To optimize the delivery rate of polysaccharide via nebulizer, the volumetric flow rate (L/min) of the nebulizer preferably does not exceed two times the patient's minute ventilation, although this can be varied depending on the polysaccharide formulation and the clinical status of the patient. This is because the average inspiratory rate is about twice the minute ventilation when exhalation and inhalation each represent about half of the breathing cycle. In one mode of the invention, a nebulizer with a volumetric flow rate of under 15 L/min is employed.

The particle size distribution generated from nebulizers is a function of a number of variables related to the nebulizer as well as the formulation (as discussed above). Nebulizer related factors for compressed air nebulizers include air pressure, air flow, and air jet diameter. Nebulizer related factors for ultrasonic nebulizers include ultrasound frequency, and rate/volume of air flow. In one mode of the invention, a compressed air nebulizer with specific air pressure, air flow, and hole diameter settings is used to generate droplets of a specific polysaccharide formulation under 10 microns. In another mode, an ultrasonic nebulizer with specific frequency and hole diameter settings is employed to generate droplets of a specific polysaccharide formulation under 10 microns.

Other considerations that determine selection of an ideal nebulizer and formulation include solution use rate (ml/min), aerosol mass output (mg/L), and nebulizer “hold up” (retained) volume (ml). The interaction among these factors will be appreciated by those of skill in the art.

Aerosolized polysaccharide could be delivered from nebulizer to a patient's respiratory tract via face mask, nonrebreather, nasal cannula, nasal covering, “blow by” mask, endotracheal tube, and Ambu bag. All of these connections between the patient and nebulizer are considered to fall within the scope of the present invention.

Given that this invention is a nontoxic therapy, which exerts its beneficial effects in respiratory disease by its physical presence in the lung, the formulation of this invention should allow for the polysaccharide to remain in the lung continuously. The half-life of HA injected in the pleural (potential space between the lung and the chest wall) of rabbits has been shown to range between 8 and 15 hours. The half-life is longer if more HA is injected. Commonly inhaled medications for emphysema are used from one to three times a day. More frequent dosing requirements present a compliance issue with patients. One aspect of this invention involves a formulation of polysaccharide that resides in the lung for 6 hours to be given 4 times per day, or preferably for 8 hours, to be given three times per day. A more preferable embodiment is a formulation that remains in the lung for 12 hours, which will be administered twice a day. A more preferable embodiment is a formulation that remains in the lung for 24 hours, which will be administered once a day.

The effect of different formulations on duration is studied in mammals by tagging the polysaccharide with a radiolabel such as tritium, C¹⁴, Thallium, or Technecium. Alternatively, a direct assay for the particular polymer could be employed. One radiometric assay for HA uses ¹²⁵I-labeled HABP (HA binding protein); this assay is commercially available from Pharmacia (“Pharmacia HA Test”). Material is delivered to the lungs and monitored over time by use of a scintillation counter (e.g. gamma camera). Alternatively, a group of animals (e.g. rats) is given radiolabeled-glycosaminoglycan in the lungs and then serially sacrificed over time. Excised lung tissue is examined for radioactivity, and duration time or half-life is determined.

In addition to delivery of polysaccharides via nebulizer and via direct instillation through the anterior aspect of the trachea, polysaccharides could also be delivered to target lung tissue via bronchoscopy. Bronchoscopy is a procedure where pulmonary physicians insert a scope into a patient's mouth, through the trachea, and into the bronchial airways. The scope allows visualization and access into the lungs for diagnosis (e.g. collection of bronchial alveolar lavage samples) and therapeutic procedures (e.g. placement of stents). One mode of the invention involves delivery of polysaccharides via a bronchoscope to specific regions of the lung.

Another mode of the invention involves transthoracic delivery of polysaccharides. Polysaccharides can be delivered into the pleural space either percutaneously through a needle or via a catheter or chest tube. This pleural space application might benefit patients with pain from pleurisy, metastases, adhesions, pneumothorax, or pulmonary embolism. Given that polysaccharides and glycosaminoglycans in particular enhances healing, injection of glycosaminoglycans into the pleural space might quicken the healing process of patients with a pneumothorax (collapsed lung). In addition, the viscoelastic properties of glycosaminoglycans might enhance elastic recoil of the lungs.

In addition to delivery via unassisted inhalation, another embodiment of the invention involves delivery of aerosolized polysaccharides under positive pressure ventilation. A commonly used ventilatory assist device is CPAP: Continuous Positive Airway Pressure. In this application, a breathing mask is sealed around the mouth of a patient. The patient is then administered oxygen through the mask at a certain pressure to facilitate inspiration. Delivery of polysaccharides through a CPAP mask might enhance delivery of material to the deep airways. To facilitate delivery to the alveoli and transfer across the alveolar epithelial barrier, the polysaccharide could be delivered while the patient is being ventilated with positive end expiratory pressure (PEEP).

Another mode of the invention is to deliver aerosolized polysaccharides with a device that delivers material when the patient generates a certain level of negative inspiratory pressure.

Another mode of the invention is to deliver polysaccharides in conjunction with ventilation through an endotracheal tube. One benefit of this embodiment is to protect against oxygen toxicity in patients ventilated with high concentrations of oxygen. In addition the viscoelastic properties of polysaccharides should protect the lungs from ventilator associated barotrauma that results in the complication of pneumothorax.

Polysaccharides could be delivered through the endotracheal tube in such a fashion as to serve as a protective coating between the endotracheal tube (either the distal end or the cuff) and the trachea. This would reduce the incidence of tracheal stenosis, a complication of prolonged intubation.

In another aspect of the invention, methods and formulations that include a polysaccharide are disclosed for the delivery of drugs or other agents (e.g. imaging agents) to the lung for local or systemic therapies. The invention also includes methods and formulations to deliver polysaccharides to the lung before or after delivery of a drug to enhance the efficacy of the drug, in an unaltered form as a depot for slow release of drugs, in unaltered form as a drug carrier, or in an altered form as a drug conjugate.

Just as the invention encompasses protecting the lungs with aerosol polysaccharide, the invention also encompasses application of polysaccharide by aerosol delivery to other tissues, including for example, exposed tissues during surgery, sinus passageways, burns, and mucous membranes.

Polysaccharides may be delivered to the lung for slow release via encapsulation or carrier materials such as liposomes, or other drug “shells” such as albumin (Albunex by Molecular Biosystems), sugars (Levovist by Schering), gelatins, or lipids.

Specific embodiments of one aspect of the invention, wherein the polysaccharides are used in conjunction with and/or to facilitate delivery of a second agent are now described in detail with respect to HA, one preferred polysaccharide in accordance with the present invention.

Unmodified HA may be combined with drugs for delivery to the lungs. Unmodified HA has been used as a drug carrier in ophthalmic use (pilocarpine), to enhance absorption of drugs and proteins through mucous tissues, to enhance the activity of drugs (non-steroidal anti-inflammatory drugs (NSAIDs), cyclosporin), and to serve as a drug reservoir or “depot” for slow release of drugs (diclofenac). Unmodified HA could be combined with peptides such as insulin to enhance absorption through the lungs into the systemic circulation. Unmodified HA could serve as a “depot” for slow release of drugs targeting the lung (see e.g., 1), or as a “depot” for slow release of drugs intended for systemic delivery (e.g. narcotics, insulin, other naturally occurring peptides).

HA receptors are overexpressed in metastatic cancer cells. This could offer opportunities to deliver targeted anticancer agents to lung cancers via an HA carrier.

HA has been esterified for attachment of NSAIDs and steroids (methylprednisolone). Other HA derivatives have been described for attachment of drugs, including hydrazide modification of HA to carry NSAIDs and steroids. Antibiotic compounds such as doxorubicin have been attached to HA via an amide bond. Acytylated HA has been coupled with anticancer drugs such as 5FU and cytosine arabinoside. These and other HA-drug conjugates could be used for delivery via aerosol of compounds to the lung, particularly the lung matrix where HA binds to elastin fibers. These HA-drug conjugates could deliver lung therapeutics (see 1), or systemic agents.

HA could be bound to imaging agents such as nuclear tags (e.g. Thallium) or contrast dyes for inhalation to the lung. Since HA binds to elastin fibers, this would permit imaging of the lung matrix.

Local delivery of drugs to the lung has been used in the treatment of respiratory diseases such as asthma and in protein therapies such as DNase for cystic fibrosis. The deep part of the lung, which contains the alveoli has a large surface area, thin tissue lining and limited number of proteolytic enzymes, which is certainly advantageous for systemic delivery of pharmaceuticals.

Most current lung delivery systems deliver drugs in liquid forms, preferably by pushing liquid drug formulations through very tiny nozzles (2.5 micron diameter) at inspiratory flow rate and inhaled volume.

Fine dry-powder can also be delivered to the lung as an aerosol cloud. This is generated by compressing air into the drug powder inside the inhaler, thus dispersing the powder into a cloud of tiny particles (1-5 micron) that are capable of reaching the deep part of the lung. These newer inhalers reproducibly deliver 20-50-% of the drug to the lung.

Drug Delivery

Polysaccharides and their derivatives can be formulated with drugs as a liquid or solid form, and can be nebulized or aerosolized and delivered to the lung. Once delivered to a specific tissue site such as the lung, drugs are released from the polysaccharide through various mechanisms.

1. Polysaccharide delivered as a powder swells in body fluid to form a hydrogel, which releases the associated drugs via solvent activation. Polysaccharide hydrogels are the products of chemical crosslinking of polysaccharide. High molecular weight linear polysaccharide (unmodified) can also swell significantly and is therefore useful for certain applications. The swelling is less than with the crosslinked hydrogel.

2. Erosion of polymer matrix (embedded with drugs) through a chemical reaction leading to the drug released by diffusion. The water insoluble matrices of polysaccharide are the products of hydrophobic modification or crosslinking or both.

3. Drug release after the cleavage of its covalent bonding to the polymer matrix system. Polysaccharide drug conjugates using hydrolyzable linkage form the delivery system, wherein the drug is released by hydrolysis of the linkage.

Polysaccharide deriviatives may be used to enhance drug delivery. For example, crosslinked HA may be delivered to the lung alone as previously described for native HA. In addition, crosslinked HA may be delivered with other therapeutic agents. Examples of crosslinked HA derivatives and methods of making same are presented below.

HA Crosslinked by Biscarbodiimides

U.S. Pat. No. 5,356,883, to Kuo et al., entitled “Water-insoluble derivatives of hyaluronic acid and their methods of preparation and use” discloses a method for preparing water-insoluble biocompatible gels, films and sponges by reacting HA with biscarbodiimide. The final products are HA acylurea. This patent is incorporated in its entirety herein by reference thereto.

HA Crosslinked by Divinyl Sulfone

U.S. Pat. No. 4,605,691, to Balazs, et al., entitled “Cross-linked gels of hyaluronic acid and products containing such gels” teaches a method of preparing a cross-linked gel of HA, comprising subjecting HA in a dilute aqueous alkaline solution at a pH of not less than about 9 to a cross-linking reaction with divinyl sulfone at about 20° C. This patent is incorporated in its entirety herein by reference thereto.

HA Crosslinked by Di-epoxide

U.S. Pat. No. 4,863,907, to Sakurai et al., entitled “Crosslinked glycosaminoglycans and their use” discloses crosslinked glycosaminoglycans or salts thereof prepared by crosslinking glycosaminoglycan or salts thereof with a polyfunctional epoxy compound, wherein a crosslinking index is 0.005 or more per 1 mole of repeating disaccharides in glycosaminoglycan. The compounds have various medical and cosmetic uses. The polyfunctional epoxy compound may be epichlorohydrin or epibromohydrin. This patent is incorporated in its entirety herein by reference thereto.

U.S. Pat. No. 4,716,224, to Sakurai et al., entitled “Crosslinked hyaluronic acid and its use” discloses compounds similar to U.S. Pat. No. 4,863,907, wherein the polyfunctional epoxy compound is selected from the group consisting of halomethyloxirane compounds and a bisepoxy compound is selected from the group consisting of 1,2-bis(2,3-epoxypropoxy) ethane, 1,4-bis(2,3-epoxypropoxy) butane, 1,6-bis(2,3-epoxypropoxy) hexane. This patent is incorporated in its entirety herein by reference thereto.

HA Crosslinked by Multi-valent Cations

U.S. Pat. No. 5,532,221, to Huang et al., entitled “Ionically crosslinked carboxyl-containing polysaccharides for adhesion prevention” discloses a method of reducing post-operative adhesion formation by topically applying an ionically crosslinked carboxyl-containing polysaccharide or a pharmacologically acceptable salt thereof, e.g. sodium hyaluronate crosslinked with ferric chloride, to a site of surgical trauma. This patent is incorporated in its entirety herein by reference thereto.

HA Crosslinked by Dihydrazides

U.S. Pat. No. 5,652,347 to Pouyani et al., entitled “Method for making functionalized derivatives of hyaluronic acid” teaches hyaluronate finctionalized with dihydrazide, which may be cross-linked. A method for producing hyaluronate finctionalized with dihydrazide includes mixing hyaluronate and dihydrazide in aqueous solution, then adding carbodiimide so that the hyaluronate and dihydrazide react to form functionalized hyaluronate. The degree of HA crosslinking vs. HA conjugation depends upon the stoichiometry of the dihydrazide and HA. This patent is incorporated in its entirety herein by reference thereto.

HA Crosslinked by Phosphorus Compounds

U.S. Pat. No. 5,783,691, to Malson et al., entitled “Crosslinked hyaluronate gels, their use and method for producing them” teaches a crosslinked hyaluronic acid derivative in which the crosslinking has been achieved by means of reaction with a phosphorus-containing reagent, especially a derivative of an acid of phosphorus. The invention also relates to the methods of producing such a product as well as its use as a slow release depot for administration of HA or a medicament incorporated in the gel. A process for preparing gels of crosslinked sodium hyaluronate is also disclosed, which comprises reacting a solution of the sodium hyaluronate with a phosphorus acid derivative selected from the group consisting of a phosphorus acid halide, a phosphorus acid oxyhalide and a phosphorus acid anhydride under crosslinking conditions. This patent is incorporated in its entirety herein by reference thereto.

Other polysaccharide modifications are also included within the scope of the present invention. Examples of these include:

HA Modified by Designed Carbodiimides

Kuo et al., Bioconjugate Chemistry, 1991, 2:232-241 disclose HA modified by designed carbodiimides, wherein an amine functionalized HA was synthesized, to which various drugs can be attached. Incorporated in its entirety herein by reference thereto.

Carboxylate-containing chemicals such as anti-inflammatory drugs can be converted to the corresponding N-hydroxysuccinimide (NHS) active esters, which can react with the primary amine under physiological conditions. Amine-containing drugs such as peptides can be linked to the amine tether via the following approach. A thiol cleavable crosslinker such as dithiobis(succinimidyl) propionate (DSP) is used to crosslink the amine tethers of HA. The sulfhydryl groups produced through the reduction of the disulfide bonds can then react with the amino group of lysine of the peptides through the heterobifunctional crosslinker N-succinimidyl-3-(2-pyridyldithio)propionate (SPDP).

Steroid Compounds have Hydroxyl Groups and can Form Esters with HA

The methods of preparing the esters is described in U.S. Pat. No. 4,965,353. The patent describes treatment of selected alcohols in the presence of catalyzing substances, such as strong inorganic acids or ionic exchangers of the acid type or with an etherifying agent capable of introducing the desired alcoholic residue in the presence of inorganic or organic bases. Any etherifying agents known in literature may be used, such as in particular, the esters of various inorganic acids, including hydracids, that is hydrocarbyl halogenides, such as alkyl halides. The HA esters may, however, be prepared to advantage according to the second method, which consists of treating a quaternary ammonium salt of acid polysaccharide containing carboxyl groups with an etherifying agent. This patent is incorporated in its entirety herein by reference thereto.

U.S. Pat. No. 5,336,767 to della Valle et al., entitled “Total or partial esters of hyaluronic acid,” discloses a group of steroid compounds as possible HA drug conjugates. A total or partial ester of HA is also disclosed, with an alcohol selected from the group consisting of cortisone, hydrocortisone, prednisone, prednisolone, fluorocortisone, dexamethasone, betamethasone, corticosterone, deoxycorticosterone, paramethasone, flumethasone, fluocinolone, flucinolone acetonide, fluprednylidene, clobetasol, and beclomethasone. This patent is incorporated in its entirety herein by reference thereto.

Other drugs containing hydroxyl groups, such as the bronchodilators like ipratropium and albuterol may also be included in the above group of steroid conjugates.

Hydrazide Modification Chemistry

HA may be first modified with adipic dihydrazide (ADH), and the remaining pendant hydrazide groups are coupled to NHS esters of ibuprofen or hydrocortisone hemisuccinate at pH 8.2 as detailed by Pouyani et al., Bioconjugate Chem, 1994, 5:339-347. Incorporated in its entirety herein by reference thereto.

Cyanogen Bromide Activation Method

This reaction between HA and BrCN leads to the attachment of amine containing drugs via a urethane bond to one of the hydroxylic finctions of HA. The examples of drugs with amino groups are anthracycline antibiotics adriamycin and daunomycin, as disclosed by Cera et al., Int. J. Biol. Macromol., 1988, 10:66-74. Incorporated in its entirety herein by reference thereto.

Sodium Periodate Oxidation Method

Reactive aldehydes can be generated from the vicinal secondary alcohol functions on HA. The aldehyde then reacts with primary amines containing molecules such as peptides to form the conjugates as taught by Glass et al., Biomaterials, 1996, 17:1101-1108. Incorporated in its entirety herein by reference thereto.

Polysaccharides may also be derivatized to enhance their effectiveness as drug carriers/conjugates. Examples of derivatized HA are provided below.

HA Total or Partial Esterification

U.S. Pat. No. 5,202,431, to della Valle et al., like the previous della Valle patent, teaches esterification wherein the alcohol moieties are not pharmaceutically active. Examples are partial esters of HA with an alcohol of the aliphatic, araliphatic, cycloaliphatic or heterocyclic series wherein at least a first portion of the carboxylic acid groups of said hyaluronic acid are salified with a therapeutically active amine. The compounds possess bioplastic and pharmaceutical properties and may be used in innumerable fields, including cosmetics, surgery and medicine. This patent is incorporated in its entirety herein by reference thereto.

The therapeutically active amines include all the nitrogenized and basic drugs such as those included in the following groups: alkoloids, peptides, phenothiazines, benzodiazepines, thioxanthenes, hormones, vitamins, etc. See also: Langer, “Drug Delivery and Targeting”, Nature, 1998, 392[supp]:5-10, and Vercruysse et al., “Hyaluronate derivatives in drug delivery”, Critical Reviews in Therapeutic Drug Carrier Systems, 1998, 15(5):513-55. Incorporated in its entirety herein by reference thereto.

The structural features for the following drugs for pulmonary use are described in 1. For carboxyl, amino or hydroxyl functionalities of the drugs, conjugations using the methods described in Section (C) above are possible choices. Most of the drugs listed have significant hydrophobic property and can be trapped in crosslinked or modified HA with increased hydrophobicity.

| | generic | structural features |
|---|---|---|
| Inhaled Steroids | beclomethasone | hydroxyl, hyrophobic |
| (anti- | budesonide | hydroxyl, hyrophobic |
| inflammatory) | flunisolide | hydroxyl, hyrophobic |
| | triamcinolone | hydroxyl, hyrophobic |
| | acetonide | |
| Beta Agonists | albuterol | hydroxyl, hyrophobic, nitrogenized |
| (bronchodilator) | isoetharine | hydroxyl, hyrophobic |
| | metaproterenol | hydroxyl, hyrophobic, nitrogenized |
| | pirbuterol | hydroxyl, hyrophobic, nitrogenized |
| | salmeterol | hydroxyl, hyrophobic, nitrogenized |
| | terbutaline | hydroxyl, hyrophobic, nitrogenized |
| | epinephrine | hydroxyl, hyrophobic, nitrogenized |
| Anticholinergics | ipratropium | hydroxyl, hyrophobic, nitrogenized |
| | bromide | |
| Mast Cell | cromolyn sodium | carboxyl, hydroxyl, hyrophobic, |
| Stabilizer | nedocromil | carboxyl hydroxyl, hyrophobic, |
| | | nitrogenized |
| Leukotriene | montelukast | carboxyl, hyrophobic, nitrogenized |
| Inhibitors | zafirlukast | hyrophobic, nitrogenized |
| | zileuton | hyrophobic, nitrogenized |
| Methylxanthines | theophylline | nitrogenized |
| (bronchodilator) | aminophylline | nitrogenized |
| Surfactants | beractant | hydrophobic |
| | colfosceril | hydrophobic, nitrogenized |
| | palmitate | |
| Mucolytic | acetylcysteine | carboxyl, nitrogenized |
| Cystic Fibrosis | “Pulmozyme” | DNase or domase alpha |
| drug | | (Genentech) |
| | P2Y2 receptor | (Inspire Pharm.) |
| | agonists | |
| | (INS365) | |
| Antimicrobials | Penicillins, | carboxyl, hyrophobic, nitrogenized |
| | Cephalosporins, | carboxyl, hyrophobic, nitrogenized |
| | Sulfonamides | nitrogenized, hydrophobic |
| | Tetracylcines | hydrophobic, nitrogenized, |

All the following nonsteroids have carboxyl and are hydrophobic

| | | |
|---|---|---|
| Nonsteroidals | Salicylate Class: | aspirin |
| | Propionic acids: | ibuprofen, naproxen |
| | Acetic acids: | indomethacin, ketorolac |
| | Fenamates: | meclofenamate |
| | Oxicams: | peroxicam |
| | Cox-2 inhibitors: | celecoxib, rofercoxib |

Anti-cancer agents

| | | |
|---|---|---|
| Alkylating agents | cisplatin, | metalic, nitrogenized |
| | cyclo- | nitrogenized, hydrophobic |
| | phosphamide | |
| Antimetabolites: | fluorouracil, | nitrogenized |
| | methotre- | hydrophobic, nitrogenized |
| | xatecarboxyl, | |
| Mitotic | paclitaxel | hydrophobic, nitrogenized, |
| | (Taxol), | |
| Inhibitors: | vincristine | hydrophobic, nitrogenized |
| Immuno- | interferon | nitrogenized, hydrophobic, |
| modulators: | | carboxyl (as glycoprotein) |

Elastase Inhibitors

| | |
|---|---|
| Naturally-occurring: | alpha 1 antitrypsin |
| Synthetic: | |
| inhibitors of neutrophil: | methyl ketone derivatives |
| elastase | |
| inhibitors of macrophage: | RS113456 |
| metalloproteinase | |
| elastase inhibitor: | ABT-491 (Abbot) |
| HNE inhibitor: | Ono-5046 (Ono) |
| Alpha 1-Antitrypsin: | Recombinant AT-1 (Novartis) |
| Elastase inhibitor: | Erdosteine (Edmond Pharma) |
| Elastase inhibitor: | FK-706 (Fujisawa) |
| A1-AT agonist: | Gene Active AT-1 (Gene Medicine) |
| Elastase inhibitor: | Midesteine (Medea) |
| Proteinase inhibitor: | CMP-777 (Dupont) |
| HNE inhibitor: | CE-1037 (Cortech/United Ther) |
| HNE inhibitor: | CE-2000 series (Cortech/Ono) |
| HNE inhibitor: | EPI-HNE-4 (Dyax) |
| HNE inhibitor: | MDL-101146 (HMR) |
| HNE inhibitor: | EPI-HNE-1 (Protein Engineer) |
| Cathepsin G inhib.: | LEX-032 (Sparta) |
| HNE inhibitor: | WIN-63759 (Sterling Winthrop) |
| HNE inhibitor: | SPAAT (UAB Res. Found.) |
| HNE inhibitor: | ZD-8321 (AsiraZeneca) |
| Recomb. inhibitor: | SLP-1 (Amgen) |
| Elastase inhibitor: | GW-311616 (Glaxo-Wellcome) |
| ON-Elastase inhibitor: | NX-21909 (Gilead) |
| Elastase inhibitor: | SR-268794 (Sanoti) |
| Elastase inhibitor: | SYN-1134 (Syn. Pharm.) |
| HNE inhibitor: | ZD-0892 (AsiraZeneca) |

In accordance with one embodiment of the present invention, methods and formulations are described herein with respect to GAGs. In order to fully specify this preferred aspect of the methods and formulations, various embodiment-specific details are set forth, such as the molecular weight, concentration, viscosity, etc. of the GAG formulations. It should be understood, however, that these details are provided only to illustrate preferred embodiments of the method, and are not intended to limit the invention to the GAGs of the preferred embodiments. Indeed, although the invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

### EXAMPLES

### Example 1

### Effect of HA on Pulmonary Emphysema Induced by Pancreatic Elastase

Measurements of air-space size were performed 1 week after intratracheal instillments of elastase and HA or elastase and saline. As shown in FIG. 1, animals given 1 mg of HA immediately following elastase administration showed a marked reduction in air-space enlargement compared to those secondarily receiving saline (82 vs 122 μm). Histological examination of the lungs from both treatment groups showed minimal inflammatory changes composed of scattered intraalveolar collections of neutrophils and red blood cells. No specific changes were associated with the added administration of HA.

Animals instilled with 1 mg of HA, 2 hrs preceding elastase, had a significantly lower mean linear intercept than controls receiving saline, then elastase (96 vs 120 im; p<0.05; FIG. 1). A further reduction in airspace enlargement was seen with 2 mgs of HA, which resulted in a mean linear intercept of 88 im (p<0.05 vs controls; FIG. 1).

Instillment of 2 mgs of HA, 1 hr after elastase, also resulted in a significant decrease (p<0.05) in air-space enlargement (66 vs 104 im; FIG. 1). However, 1 mg of HA, given either 1 or 2 hrs after elastase administration, did not significantly affect the mean linear intercept (treated vs control: 100 vs 104 im at 1 hr, 114 vs 124 im at 2 hrs; FIG. 1).

These results indicate that HA ameliorates elastase-induced emphysema. Furthermore, they suggest that the protective effect of HA may involve early events in the development of the experimental injury which precede elastic fiber breakdown. It has been shown that HA has no elastase inhibitory capacity, the decrease in lung injury may possibly be related to indirect effects between the polysaccharide and elastase, such as reduction of enzyme mobility within the lung interstitium, or, alternatively, direct interactions between HA and elastic fibers themselves.

### Example 2

### Effect of HA on Emphysema Induced by Neutrophil Elastase

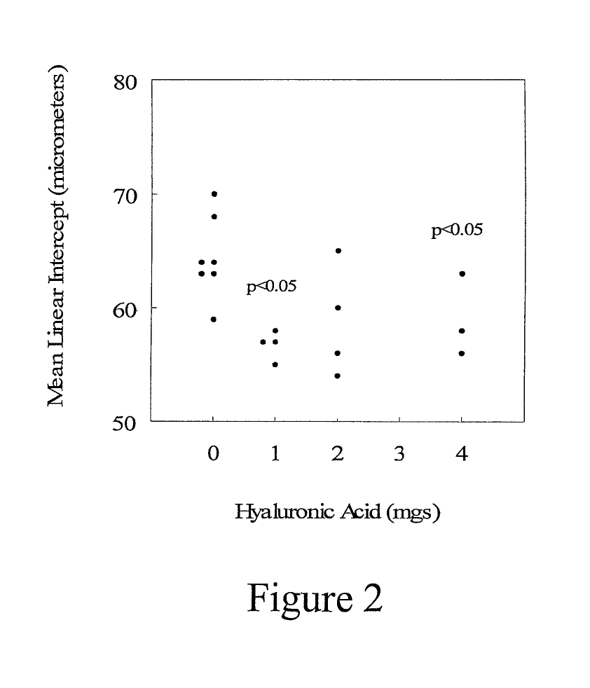

Two hours prior to intratracheal instillment of 40 units of human neutrophil elastase, animals were given either 1, 2, or 4 mgs of HA via the same route. Compared to controls receiving saline alone, all groups administered HA showed a decreased mean linear intercept (FIG. 2). The values were significantly lower (p<0.05) in animals receiving 1 and 4 mgs of HA (57 and 59 im, respectively, vs 64 im for controls). In contrast to pancreatic elastase-induced emphysema, there was no correlation between the amount of HA instilled and the degree of reduction in mean linear intercept. This is not surprising in view of the fact that neutrophil elastase is less effective than its pancreatic counterpart in producing air-space enlargement. The mean linear intercept measurements seen with HA treatment are close to normal values, which range from 50-60 im, based on previous determinations (Cantor et aL (1993) Exper Lung Res 19:177-192; Cantor et al. (1995) Exp Lung Res. 21:423-436).

### Example 3

### Effect of HA on Elastase Activity

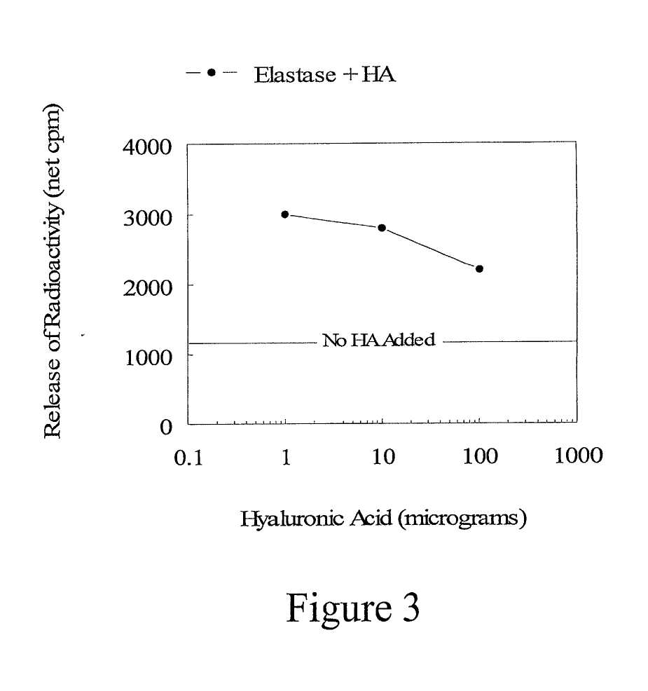

Incubation of HA with pancreatic elastase did not reduce ³H-elastin breakdown, but instead caused an increase in the release of radioactivity from the substrate (FIG. 3). This stimulatory effect may result from greater interaction between enzyme and substrate (possibly due to alteration of electrostatic bonding).

Characterization of HA Preparation

The average molecular weight of the commercial bovine tracheal HA used in all the experiments described above was 104,800, based on intrinsic viscosity measurements (Table 1).

TABLE 1
Chemical and Physical Characteristics of Bovine tracheal HA

| | | | Intrinsic | |
|---|---|---|---|---|
| Uronic Acid | Hexosamine | | Viscosity | |
| (ig/ml) | (ig/ml) | Protein (%)* | (cc/gm) | M.W. (Daltons) |
| 94.0 | 93.8 | 4.6 | 292 | 104,800 |

Ratio of UA/Hexosamine = 1.0
*Percentage of protein calculated on the basis of HA content.

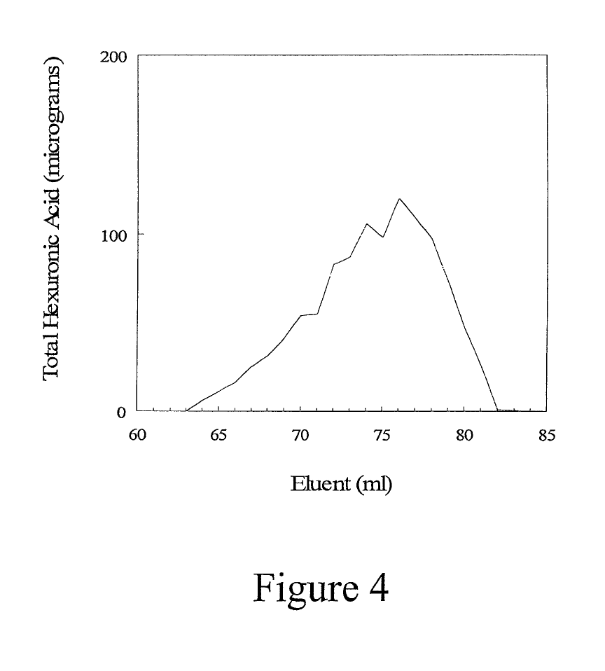

This value is relatively low compared to other preparations of HA, some of which may have molecular weights in excess of 3×10⁶ Daltons. The material tested was relatively pure, containing less than 5 percent protein, and the uronic acid to hexosamine ratio was 1.0, which is characteristic of HA. Gel filtration chromatography revealed a broad elution profile (FIG. 4), containing polysaccharide chains of varying lengths, a feature commonly observed with HA preparations.

### Example 4

### Preparation of Fluorescein-labeled HA

Fluorescein amine was coupled to bovine tracheal hyaluronic acid, according to previously published techniques (Anthony et al. (1975) Carbohydrate Res. 44: 251-257). A solution of 100 mgs of HA in 80 ml water was diluted with 40 ml dimethyl sulfoxide and combined with acetaldehyde (50 ìl), cyclohexyl isocyanide (50 ìl), and fluorescein amine (50 mgs). The mixture was incubated at 22° C. for 5 hrs and the resultant fluorescein-labeled HA was isolated by alcohol precipitation and gel filtration. Thin-layer chromatography was used to determine the purity of the preparation.

### Example 5

### Studies Using Fluorescein-labeled HA

Female Syrian hamsters, weighing approximately 100 gms each, were instilled intratracheally with 2 mgs of the fluorescein-labeled HA (in 0.2 ml saline solution), according to procedures described above. At 1, 2, 4, 24, and 72 hrs following instillment, the animals were sacrificed and their lungs were prepared for histology. Unstained slide sections were then prepared and subjected to fluorescence microscopy. Sections were also stained for elastic fibers (Verhoeff-Van Gieson stain) and examined with a light microscope.

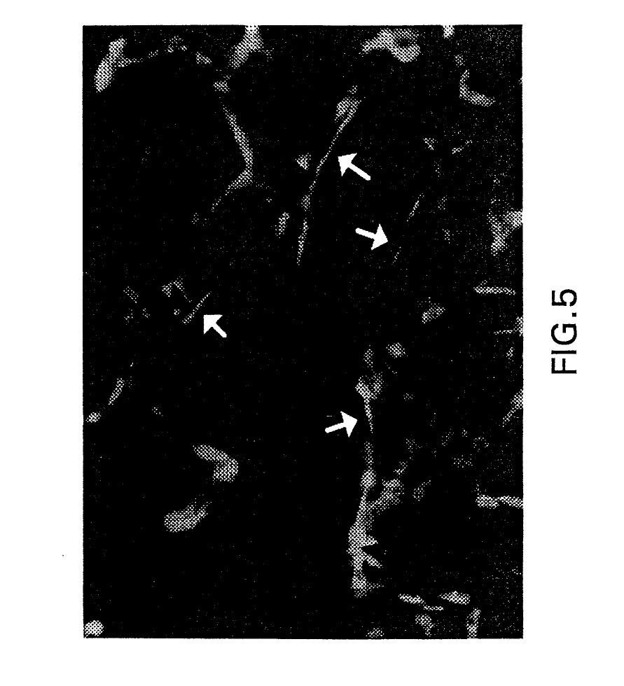

Fluorescence microscopy revealed a rapid influx of labeled HA into the lung. Since the labeled HA was instilled intratracheally, its distribution was patchy. At 1, 2 and 4 hrs, there was prominent fluorescence associated with interstitial, pleural, and vascular elastic fibers (FIGS. 5,6). The identity of these fibers was confirmed with the Verhoeff-Van Gieson elastic tissue stain. Alveolar macrophages, which rapidly sequestered the labeled HA, also showed strong fluorescence.

By 24 hrs, overall fluorescence was significantly reduced, and much of the specificity for elastic fibers was missing. Alveolar macrophages, however, remained strongly fluorescent, even at 72 hrs.

The fluorescence associated with elastic fibers suggests that the lung may be protected from elastase injury by the temporary coating of these fibers with the instilled HA. This process appears to occur quickly and extend for at least 4 hours, explaining why air-space enlargement can be decreased by instilling HA either 2 hrs before or 1 hr after elastase administration (FIG. 1). The lack of protection observed when HA was instilled 2 hrs after elastase suggests that significant damage to elastic fibers may have occurred by this time (FIG. 1).

### Example 6

### Aerosolization of HA

Fluorescein-labeled HA (0.1 percent solution in water) was administered to hamsters using a nebulizer. After exposure to the aerosol for 50 minutes, the animals were sacrificed. Fluorescent microscopy of the lungs showed a more uniform distribution of fluorescent elastic fibers than that seen with intratracheally instilled fluorescein-HA, above. Furthermore, the aerosolized HA showed a protective effect against neutrophil elastase. Animals treated with an aerosol composed of 0.1% HA in water for 50 minutes, then instilled -intratracheally with 80 units of neutrophil elastase, had a significantly lower mean linear intercept than controls treated with aerosolized water alone (68.2 im vs 85.9 im; p<0.05).

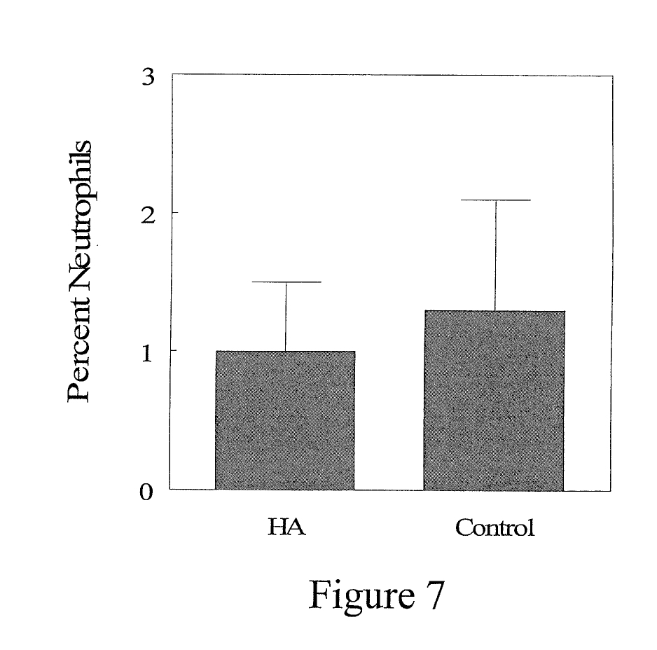

Possible inflammatory changes resulting from the aerosolized HA were determined by measuring the percentage of neutrophils in bronchoalveolar lavage fluid at 24 hours. Animals receiving HA showed no difference from controls exposed to aerosolized water for a similar time period (FIG. 7).

### Example 7

### Prevention of Elastic Fiber Damage in Vitro

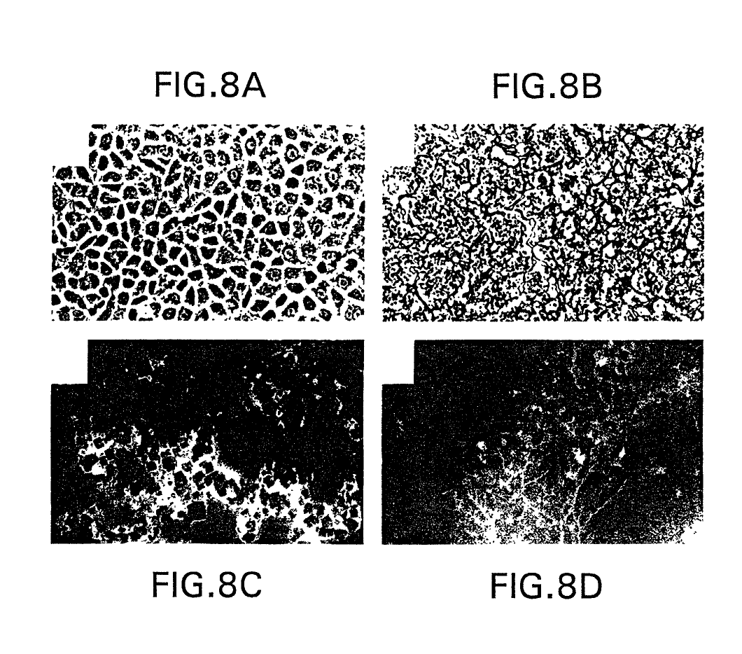

Since HA has no elastase inhibitory capacity, the mechanism responsible for its protective effect needs to be clarified. To address this issue, radiolabeled extracellular matrices, derived from cultured rat pleural mesothelial cells, were treated with HA and then incubated with porcine pancreatic elastase. The mesothelial cells have a polygonal appearance in culture (FIG. 8A) and produce a prominent extracellular matrix containing numerous elastic fibers (FIG. 8B). The cultures have previously been shown to synthesize abundant elastin, the primary component of these fibers. Radiolabeled matrices are prepared by incubating the cultures with ¹⁴C-lysine, then lysing the cells and removing them from the culture, leaving the residual extracellular matrix intact.

As shown by fluorescence microscopy (FIG. 8C), fluorescein-labeled HA binds to the mesothelial cell matrix. Following exposure of the matrices to porcine pancreatic elastase (100 ng/ml) for 1 hr, much of the fluorescein-HA is removed, but the remaining fluorescence indicates that the matrix is largely intact (FIG. 8D). The loss of fluorescence suggests that HA is specifically bound to elastic fibers.

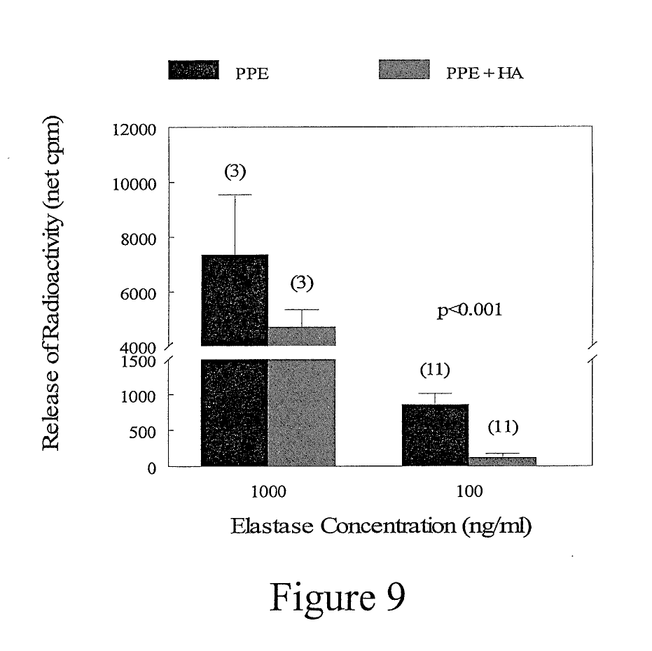

To determine if HA protects elastic fibers from injury, radiolabeled matrices were treated with 1 mg/ml of fluorescein-HA for 10 min, then incubated with either 1 μg/ml or 100 ng/ml elastase for 1 hr (FIG. 9). While release of radioactivity was reduced by HA at both concentrations of elastase, there was a much greater protective effect with 100 ng/ml of enzyme (855 vs 117 cpm; p<0.001). These results indicate that the loss of fluorescence following elastase treatment (FIG. 8D) is associated with minimal degradation of elastic fibers, suggesting that HA is only superficially bound to these fibers. It is unlikely that HA undergoes direct breakdown, since it is not a substrate for pancreatic elastase.

### Example 8

### Testing the Efficacy of a Second Preparation of HA

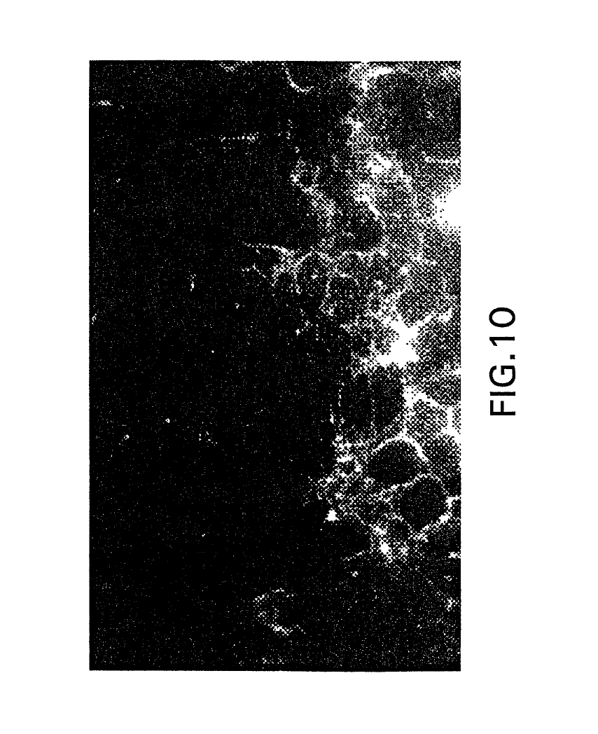

To determine if other forms of HA have a protective effect similar to the bovine tracheal preparation, a second form of HA was tested in vitro, using rat pleural mesothelial cell matrices. Streptococcal HA, produced by fermentation, was chemically modified to reduce its average molecular weight to approximately 100,000 Daltons (similar to the bovine tracheal HA used in all previous experiments). The new material was then conjugated to fluorescein as described above and tested for its ability to coat mesothelial cell elastic fibers. Fluorescence microscopy revealed a pattern similar to that seen with the bovine tracheal HA preparation (FIG. 10), demonstrating that other forms of HA may be equally effective in coating elastic fibers from injury.

In a previous study from this laboratory, in which hyaluronidase was found to synergistically interact with 60% oxygen to produce air-space enlargement, it was hypothesized that HA and other glycosaminoglycans may protect elastic fibers. Several studies support this concept by providing evidence that HA is closely associated with elastic fibers. Degradation of HA might therefore be necessary for elastases and cells, such as monocytes or neutrophils, to gain access to these fibers. As shown in a previous study from this laboratory, pretreatment of the lung with hyaluronidase resulted in an additional significant increase in airspace enlargement over that induced by intratracheal instillment of elastase alone.

The studies described above provide additional evidence that HA forms a complex with elastic fibers. The strong association of the fluorescein-labeled HA with elastic fibers clearly indicates that the instilled HA coats these fibers. Furthermore, studies using radiolabeled mesothelial cell matrices demonstrate that coating the elastic fibers with HA protects them from injury by elastase.

It has been shown that a loss of HA can reduce extravascular water content in the lung interstitium. Negatively charged carboxyl groups attached to the saccharide moieties repel one another, enlarging the domain of HA and enhancing its ability to entrap water. The hydrated and expanded HA may protect alveolar elastic fibers from contact with elastase.

The studies described above also addressed the question of whether HA is effective against neutrophil elastase, which has access to the lung parenchyma through neutrophil migration and secretion, as well as macrophage sequestering of the enzyme. In previous experiments, the use of intratracheally instilled HA was only tested against porcine pancreatic elastase, which experimentally produces more air-space enlargement than neutrophil elastase, but is not involved in the pathogenesis of human emphysema. The fact that HA is effective against neutrophil elastase increases the possibility that it may be useful in limiting alveolar damage occurring in emphysema. Furthermore, the ubiquity of neutrophil elastase in various lung inflammatory reactions suggests the possibility that HA may be effective against other forms of pulmonary injury as well.

As a possible treatment for pulmonary emphysema and other diseases involving elastic fiber injury, HA and other polysaccharides should be well-tolerated by the lung and other organs. Studies from this laboratory, described above, have shown that aerosolization of HA does not cause pulmonary inflammation. Furthermore, HA has been administered to other tissues without adverse consequences. In contrast to elastase inhibitors, which are now being considered as therapeutic agents for emphysema, HA and other polysaccharides might provide a more direct form of lung protection with fewer potential side-effects.

### Example 9

### Preparation of Low-molecular Weight HA and Fluorescein Labelling

Low molecular weight (approximately 100 kD) streptococcal HA, produced by fermentation, was obtained from Glycomed Research (Hastings-on-Hudson, N.Y.). The average molecular weight of the material was determined by measuring viscosity, using a Cannon semi-micro dilution viscometer (Cannon Instruments Co., State College, Pa.). Intrinsic viscosity (9) was determined by extrapolating viscosity measurements to zero concentration. Average molecular weight was calculated by using intrinsic viscosity data in the Mark-Houwink equation, i.e., ç=K(M)^{a} where a and K are constants for HA in saline solution. The streptococcal HA had an average molecular weight of 101 kD, which is similar to that of bovine preparations.

The purity of the HA preparation was determined by measuring the content of uronic acid, hexosamine, and protein. Uronic acid was measured with the carbazole reaction method. The content of hexosamine was determined by a modification of the Elson-Morgan procedure. The ratio of hexuronic acid to hexosamines was 1: 1, which is characteristic of HA. Protein was measured by the method of Lowry et al. Protein content of the streptococcal HA preparation was less than 0.1 percent.

Fluorescein labeling of the low molecular weight HA was performed according to previously published techniques (Anthony et al. (1975) Carbohydrate Res. 44: 251-257). A solution of 100 mgs of HA in 80 ml water was diluted with 40 ml dimethyl sulfoxide and combined with acetaldehyde (50 ìl), cyclohexyl isocyanide (50 ìl), and fluorescein amine (50 mg). The mixture was incubated at 22° C. for 5 hours and the resultant fluorescein-labeled HA was purified by alcohol precipitation and gel filtration on Sephacryl S-500, using a 1×135 cm column equilibrated with 0.2 M pyridine-acetate buffer at pH 6.2. As previously demonstrated, the fluorescein labeling procedure does not significantly degrade HA.

### Example 10

### Determination of the Effect of HA on Elastase Digestion of a Cell-free Tissue Culture Matrix

Rat pleural mesothelial cells, obtained from the American Type Culture Collection (Rockville, Md.), which have previously been shown to synthesize elastin, were cultured in 75 cc plastic flasks using Nutrient Mixture Ham's F-12 medium supplemented with 15% fetal bovine serum, 1% glutamine, 20 units/ml streptomycin, and 20 units/ml penicillin G. The cultures were incubated at 37° C. in a humidified atmosphere containing 5% CO₂. Cells and extracellular matrix were radiolabeled for 6 weeks with ¹⁴C-lysine (6.25 ìCi/flask). At the end of the labeling period, the cultures were washed with phosphate-buffered saline (PBS) and the cells were lysed with 0.5% sodium deoxycholate and EGTA. Following removal of the cellular material, the matrix was rinsed with PBS and allowed to air dry. The plastic surface containing the radiolabeled matrix was then cut into 2×2 cm squares.

Both the histochemical and immunofluorescence studies demonstrate that the matrix contains a complex network of elastic fibers. Relatively little collagen is present based on the absence of positive (red) staining for this component with the Verhoeff:Van Gieson stain. Fluorescein-labeled HA binds to the matrix and produces a pattern of fluorescence which resembles the staining pattern of elastic fibers.

Radiolabeled cell-free matrix was used to determine the effect of HA on elastase-induced elastic fiber injury. The matrix squares were incubated with 0.5 mg of low molecular weight HA in 0.5 ml PBS for 30 min. at room temperature. Controls were treated with PBS alone. Following removal of the liquid, the matrices were dried, then incubated for 3 hours at 37° C. with 0.5 ml of either: 1) 10 ig/ml, 1 ig/ml or 0.1 ig/ml of porcine pancreatic elastase (Elastin Products Co., Owensville, Mo.) in 0.1 M Tris buffer, pH 8.0; 2) 10 ìg/ml of human neutrophil elastase (Elastin Products Co., Qweensville, Mo.) in 0.1 M Tris buffer, pH 8.0; or 3) 1 ìg/ml or 0.1 ìg/ml of human macrophage metalloproteinase in 0.05 M Tris buffer, pH 7.5, with 0.01 M CaCl₂ and 0.15 M NaCl. An additional set of controls was treated with Tris buffer alone under the same conditions. The liquid was then removed, combined with a single 0.5 ml PBS wash of the matrix, and measured for radioactivity in a liquid scintillation spectrometer. Release of radioactivity from the matrices was used to measure the degree of elastolysis.

Treatment of the matrices with HA reduced the amount of radioactivity released by exposure to pancreatic elastase. While there was only a small difference between HA-treated and untreated matrices with 10 ìg/ml of pancreatic elastase (3536 vs 3423 cpm), the reduction in release of radioactivity was much larger with lower concentrations of the enzyme. A 35% decrease in radioactivity was observed with 1 ìg/ml (2819 vs. 1844 cpm; p<0.01), and a 44% reduction was seen with 100 ng/ml (1257 vs 715cpm; p<0.01). Background counts from matrix treated with Tris buffer instead of elastase averaged 190 cpm.

Washing the HA-treated matrices with PBS prior to elastase treatment did not reduce the protective effect. Matrix samples treated with HA and rinsed with PBS prior to incubation with 1 ìg/ml of pancreatic elastase showed a 57% reduction in release of radioactivity compared to controls (2091 vs 833 cpm; p <0.05).

A similar protective effect was seen with human metalloproteinase. Again, the lower concentration of enzyme was associated with a greater reduction in release of radioactivity from the HA-treated matrices. A 46% decrease in radioactivity was seen with 1 ìg/ml of enzyme (855 vs. 465 cpm; p<0.01), while an 80% reduction was seen with 100 ng/ml (128 vs. 26 cpm; p<0.05). HA treatment also reduced the release of radioactivity by human neutrophil elastase. A 53% decrease in radioactivity was observed at an enzyme concentration of 10 ig/ml (990 vs. 464 cpm; p<0.001).

### Example 11

### Identification of Matrix Elastic Fibers

Immunohistochemical identification of elastic fibers within the matrix was performed, using a primary goat anti-rat lung alpha-elastin antibody (Elastin Products Co., Owensville, Mo.) and a secondary, fluorescein-labeled rabbit anti-goat IgG antibody (Zymed Laboratories, San Francisco, Calif.). Matrix samples, prepared from cells grown on glass slide cover-slips, were fixed in acetone, treated with goat serum for 30 min., and washed with PBS. The samples were then incubated with goat anti-rat lung elastin antiserum for 1 hour and again washed with PBS. After treatment with rabbit serum for 30 min., a secondary, fluorescein-labeled rabbit anti-goat IgG antibody (Zymed Laboratories, San Francisco, Calif.) was applied for 1 hr. The matrix samples were then washed with PBS, mounted on glass slides, and examined with a fluorescence microscope.

The Verhoeff-Van Gieson stain was also used to determine the presence of collagen and elastic fibers. Matrix samples, prepared from cells grown on cover slips, were fixed in 10 neutral-buffered formalin, mounted on glass slides, then stained and viewed with a light microscope.

To determine the relationship between HA and the elastic fiber network in the matrix, samples were treated with fluorescein-labeled HA (1 mg/ml) for 30 min., washed with PBS, and examined with a fluorescence microscope. The resulting pattern of fluorescence was compared to that observed with the immunohistochemical studies of the matrix elastic fibers.

### Example 12

### Aerosol Exposure to Fluorescein-labeled HA

Syrian hamsters, weighing approximately 100 g, were placed inside a dual-port plexiglass chamber and exposed to aerosolized fluorescein-HA (20 mg in 20 ml) for 50 min. via a Whisper Jet nebulizer (Marquest Medical Products, Englewood, Colo.) attached to a compressed air source. Approximately 30 min. following exposure to the aerosol, the animals were sacrificed and their lungs were fixed in situ by inserting a catheter into the trachea and instilling 10% neutral-buffered formalin at a pressure of 20 cm H₂O. After 2 hours, both the lungs and the heart were removed from the chest as a single block and additionally fixed in 10% formalin for several days. The lungs were then dissected free of extraparenchymal structures, sectioned randomly and histologically processed. Unstained slide sections were examined with a fluorescence microscope and compared to ones treated with bovine testicular hylauronidase (Poly Scientific, Bay Shore, N.Y.) to determine if the fluorescence was due to labeled HA.

Prominent fluorescence was observed after a 50-minute exposure to a 0.1% solution of the labeled HA. There was preferential adherence of the fluorescein-HA to interstitial, vascular, and pleural elastic fibers. These fibers were previously identified as elastic in nature based on comparison with tissue sections treated with the Verhoeff-Van Gieson elastic stain. Hyaluronidase treatment of the tissue sections abolished the fluorescence.

### Example 13

### Exposure of Elastase-treated Animals to Aerosolized HA

Hamsters, weighing approximately 100 g, were exposed to an aerosol solution of 20 mg HA in 20 ml water for 50 min. as in Example 4 above. Control animals were exposed to 20 ml water alone for 50 minutes. Approximately 30 min. following aerosol exposure, the animals were anesthetized with ketamine and instilled intratracheally with 40 units of porcine pancreatic elastase (Elastic Products Company, Owensville, Mo.) dissolved in 0.2 ml normal saline solution. The elastase was delivered into the trachea via a 26 gauge needles mounted on a 1 ml syringe.

One week following intratracheal instillation of HA and elastase, the animals were sacrificed by intraperitoneal injection of sodium pentobarbital. Their lungs were then fixed and histologically processed as described above. Slide sections stained with hematoxylin and eosin were coded and mean linear intercept measurements were made by an experienced morphologist.

Hamsters exposed to aerosolized HA (0.1% solution) for 50 minutes prior to intratracheal instillation of porcine pancreatic elastase had a significantly lower mean linear intercept at 1 week compared to elastase-treated animals exposed to aerosolized water alone (107.5 vs. 89.6 im; p<0.05).

### Example 14

### Long-term Aerosol Exposure Studies

Hamsters, weighing approximately 100 g, were exposed to aerosolized HA (10 mg in 10 ml water) for 25 min., 3 times a week, for 4 weeks. Seventy-two hours following the last aerosol exposure, the animals were sacrificed and their lungs were fixed in situ as described above. Slide sections of the lungs were then examined with a light microscope to determine the presence of pathological changes. Such treatment produced no morphological changes in the lung.

The two-sample t-test was used to determine statistically significant differences between treatment groups (p<0.05).

### Example 15

### Assessment of Certain Glycosaminoglycans to Protect Elastic Fibers from Digestion by Porcine Pancreatic Elastase

The assessment of the ability of a glycosaminoglyean (GAG) to inhibit the digestion of elastic fiber by elastolytic enzymes was based on a protection assay. The protected material is a natural product derived from the activities of rat pleural mesothelial cells grown in cell culture. Such cells produce an extracellular matrix that is composed principally of elastic fibers that are labeled with ¹⁴Carbon containing lysine. The radioactive label is incorporated into the matrix as the cells synthesize elastic fibers during the growth process. After growth the cells are lysed and removed from the culture. The insoluble extracellular matrix remains attached to the culture flask. Such matrix is referred to as Mesogrow-L. The elastolytic enzyme that is used as a test probe in the following assays was porcine pancreatic elastase.

Mesogrow-L has been examined by biochemical, morphologic and immunologic techniques and has been shown to be an extensive network composed mainly of elastic fibers. Such fibers are susceptible to digestion by porcine pancreatic elastase and by human neutrophil elastase. When Mesogrow-L was digested by either of these two elastases' the activity of the enzyme breaks down the insoluble elastic fibers releasing soluble radioactive fragments. Such soluble fragments were collected and quantified by liquid scintillation spectophotometry. Digestion of Mesogrow-L substrates by elastases gave a concentration response. That is, as the concentration of the enzyme was increased there was a commensurate increase in the counts per minute (CPM) as determined by liquid scintillation spectophotometry of the soluble products. Other proteolytic enzymes such as collagenases do not release appreciable soluble radioactive fragments from Mesogrow-L.

The protection assay was carried out as follows. Squares (4 cm²) covered by Mesogrow-L substrate (Mesogrow-L squares), were cut from the plastic culture vessel. Each flask yields sufficient Mesogrow-L squares to carry out one complete protection assay. Using one culture flask per assay assures the uniformity of all test material thus allowing for comparison between groups. The Mesogrow-L squares were washed with phosphate buffer saline solution (PBS) for 30 minutes and then the solution removed. The Mesogrow-L squares were divided into three groups. Four squares were used as an untreated control group (Group A) and were treated with buffers only. The second group (Group B) made up of 6 Mesogrow-L squares served as an elastase treated control group. Such group was used as a baseline against which all the protected squares were compared. The third group (Group C) consisting of 6 Mesogrow-L squares served as the protect matrix.

The assay was carried out in two steps. The first step was to expose the Mesogrow-L to buffers or to a specific material that was being examined for its protective ability. The buffer used was PBS. The following substances were tested for their protective ability: Chondroitin Sulfate A, Chondroitin Sulfate B (Dermatan Sulfate), Chondroitin Sulfate C, Heparan Sulfate, Heparin, Dextran (MW 67K avg.), Dextran (MW 160K avg.), HA (MW 227K), HA (MW 587K) and HA (MW 890K). All of the above substances were dissolved in PBS at a concentration of 1 mg/ml. The second step was buffer treatment or enzyme exposure. The buffer used was Tris Buffer, 0.2 M at pH 8. The test enzyme was porcine pancreatic elastase (PPE) (Elastin Products) dissolved in Tris Buffer. Optimum activity for this enzyme was at pH 8.

The test was carried out as follows: Mesogrow-L squares were placed in three 6 well plates, 4 in the one plate, 6 in each of the other two. Such represents Groups A, B and C respectively. Mesogrow-L squares in Group A and Group B each were covered with 0.5 ml of PBS. Mesogrow-L squares in Group C were covered with one of the 10 test substances listed above, 0.5 ml per square. All squares were incubated at room temperature for 30 minutes. Following the incubation period the buffer or test substance was removed and the squares allowed to dry. The second step was exposure to buffer or enzyme. Mesogrow-L squares in Group A were covered with 0.5 ml of Tris Buffer, squares in Groups B and C were treated with PPE in Tris buffer. All squares were incubated for 3 hours at 37° C. Following the incubation period the digest was removed from a square to a liquid scintillation vial. The Mesogrow-L square was washed with 0.5 ml of Tris Buffer and the wash added to the vial. Each subsequent test square was treated in the same fashion. The 16 vials from the test were filled with 20 ml of liquid scintillation fluid (Ecolite+, ICN) and counted in a Packard liquid scintillation counter for 20 minutes per vial.

Buffer controls (Group A) have 2 functions. First, they serve as background counts. Such counts were subtracted from those of the PPE controls (Group B) and from the GAG protected squares (Group C). Second, such controls serve to demonstrate that the buffers in which the GAGs and Elastase are solublized do not contribute to the digestion or the protective effects.

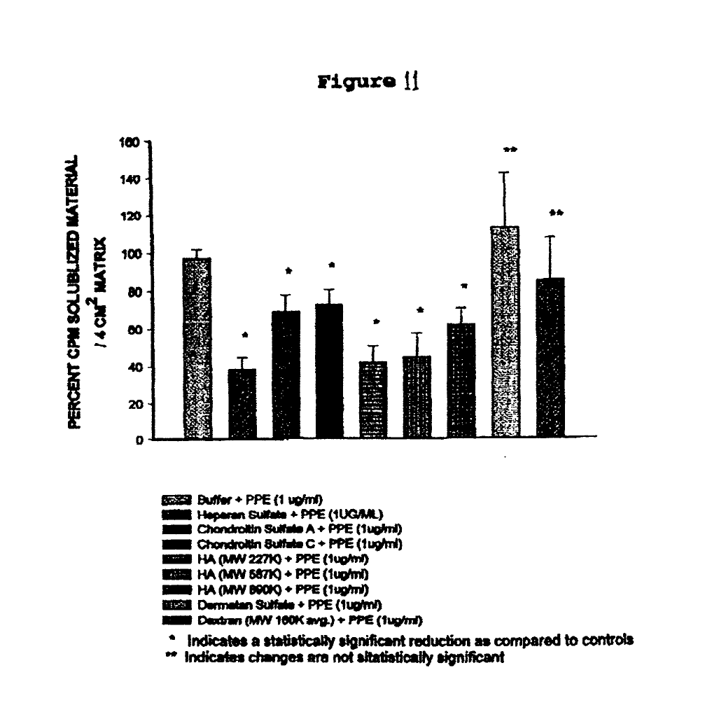

1. Chondroitin Sulfate A Assay: Chondroitin Sulfate A (Sigma) reduced the number of CPM released from a group of Mesogrow-L squares when the matrix was digested with a 1 μg/ml solution of PPE. The counts were lowered 31% overall and are statistically significant. Such a reduction in radioactive counts indicates a protective effect. See FIG. 11.

2. Chondroitin Sulfate B (Dermatan Sulfate) Assay: Chondroitin Sulfate B (Sigma) did not reduce the number of CPM released from a group of Mesogrow-L squares when the matrix was digested with a 1 ug/ml solution of PPE. Although there is a slight rise in the mean counts (FIG. 11) the statistical analysis of the data from the protected squares indicated that such counts did not vary in a statistically significant fashion when compared to the unprotected PPE treated squares.

3. Chondroitin Sulfate C: Chondroitin Sulfate C (Sigma) reduced the number of CPM released from a group of Mesogrow-L squares when the matrix was digested with 1 μg/ml solution of PPE. The counts were lowered 28% over all and were significant when compared to the control group. Such a reduction in radioactive counts indicates a protective effect. See FIG. 11.

4. Heparan Sulfate: Heparan Sulfate (Sigma) reduced the number of CPM released from a group of Mesogrow-L squares when the matrix was digested with a 1 μg/ml solution of PPE. The counts were lowered 62% overall and such results are considered statistically extremely significant. Heparan Sulfate demonstrated the greatest reduction in counts when compared to the unprotected PPE treated squares of any of the agents used in these tests. See FIG. 11 for comparison to control squares and to other tested substances.

5. HA (MW 227K) (Exhale Therapeutics): HA (MW 227K) (HA 227K) reduced the number of CPM released from a group of Mesogrow-L squares when the matrix was digested with a 1 μg/ml solution of PPE. The counts were lowered 58% overall and such results are considered statistically very significant. HA 227K had the next best overall protection when compared to the unprotected PPE treated squares after that shown by Heparan Sulfate. See FIG. 11.

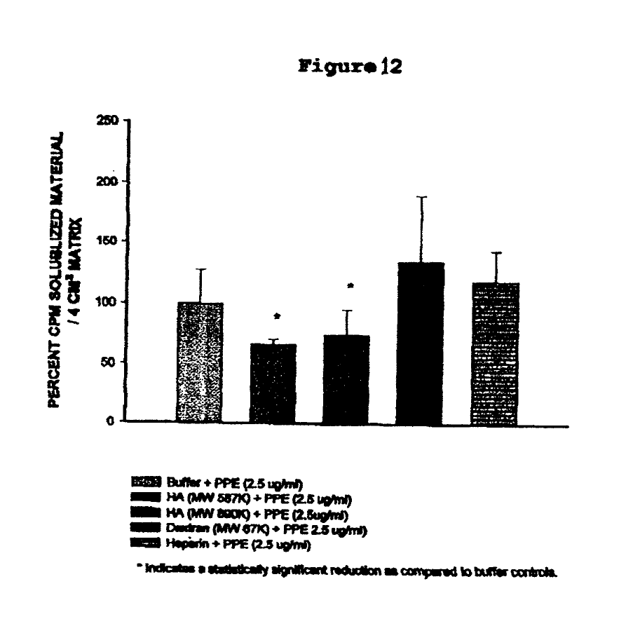

6. HA (MW 587K) Exhale Therapeutics): HA (MW 587KO (HA 587K) reduced the number of CPM released from a group of Mesogrow-L squares when the matrix was digested with a 1.0 μg/ml solution of PPE. The counts were lowered 56% overall and are statistically significant. Such a reduction in radioactive counts indicates that HA 587K is having a protective effect. See FIG. 12.

7. HA (MW 587K) (Exhale Therapeutics): HA (MW 587KO (HA 587K) reduced the number of CPM released from a group of Mesogrow-L squares when the matrix was digested with a 2.5 μg/ml solution of PPE. The counts were lowered 34% overall and are statistically significant. Such a reduction in radioactive counts indicates that HA 587K is having a protective effect. See FIG. 12.

8. HA (MW 890K) (Exhale Therapeutics): HA (MW 890K) (HA 890K) reduced the number of CPM released from a group of Mesogrow-L squares when the matrix was digested with a 1.0 μg/ml solution of PPE. The counts were lowered 39% overall and are statistically significant. Such a reduction in radioactive counts indicates that HA 890K is having a protective effect. See FIG. 12.

9. HA (MW 890K) (Exhale Therapeutics): HA (MW 890K) (HA 890K) reduced the number of CPM released from a group of Mesogrow-L squares when the matrix was digested with a 2.5 μg/ml solution of PPE. The counts were lowered 27% overall and are statistically significant. Such a reduction in radioactive counts indicates that HA 890K is having a protective effect. See FIG. 12.

10. Dextran (MW 67K avg.) (Sigma): Dextran (MW 67K avg.) (Dextran 67K) did not reduce the CPM released from a group of Mesogrow-L squares when the matrix was digested with 2.5 μg/ml solution of PPE. As with Chondroitin Sulfate B the counts were up slightly, but the statistical analysis indicated that such a rise was not significant. Dextran showed no protective effect in this test. See FIG. 12.

11. Dextran (MW 160K avg.) (Sigma): Dextran (MW 160K avg.) (Dextran 160K) did reduce the CPM released from a group of Mesogrow-L squares when the matrix was digested with 1.0 μg/ml solution of PPE. However statistical analyses of these data indicate that although there is a reduction in digestion it was not at a significant level. See FIG. 11.

12. Heparin (Sigma): Heparin did not show any protective effects. There was a slight increase in the CPM released from a group of Mesogrow-L squares when the matrix was digested with 2.5 μg/ml solution of PPE. The rise seen was not statistically different from squares treated with PPE alone. See FIG. 12.

Chondroitin Sulfate A, Chondroitin Sulfate C, Heparan Sulfate, HA 227K, HA 587K and HA 890K all demonstrated statistically significant protective effects on Mesogrow-L substrate when it was digested with porcine pancreatic elastase that was statistically significant. Of the substances tested, Heparan Sulfate seemed to have the greatest protective effect, followed by HA 227K, HA 587K, HA 890K, Chondroitin Sulfate A and Chondroitin Sulfate C. Dextran 160K also showed some overall reduction in the number of radioactive soluble products release following digestion.

The change to a higher concentration of PPE was necessitated by two factors. First the change to a new batch of Mesogrow-L. Since Mesogrow-L is a natural product each batch must be tested to check the level of radiolabel incorporated. A series of squares is tested using various concentrations of elastase to determine the optimum release of radioactive label for each batch of Mesogrow-L. Such was done but the first test was equivocal. The high concentration, 10 μg/ml, gave very high counts while the low concentration, 1 μg/ml, showed very low counts. The second factor was time. A concentration of 2.5 μg/ml was chosen to ensure that some digestions would take place during the testing otherwise no protective effect could be measured.

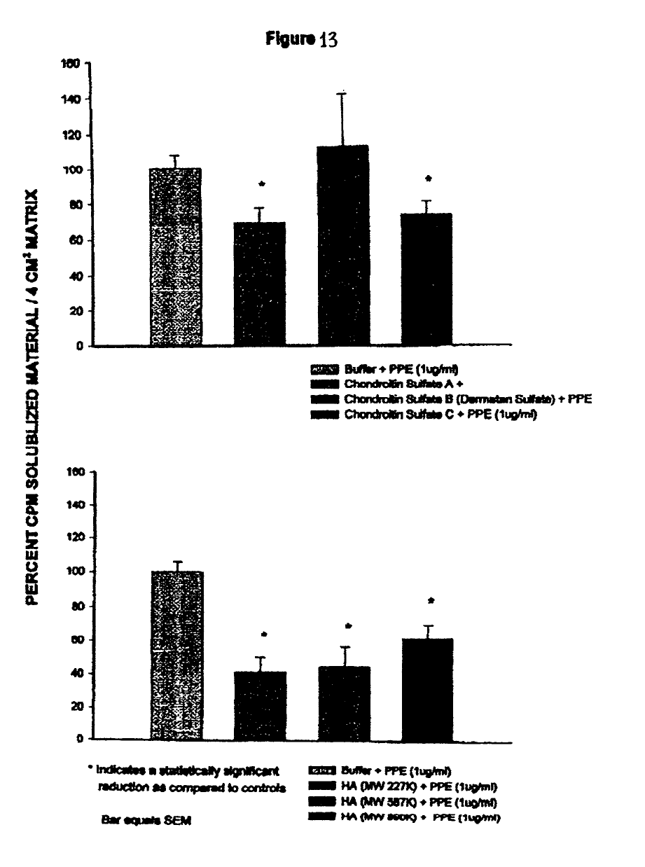

FIGS. 13a and 13b are a graphical representation of the 3 different Chondroitin Sulfates and the 3 different weight HA specimens against controls. Its interesting to note that the 2 most similar Chondroitin molecules (A & C) have a protective effect while the one that is most different does not (FIG. 13a). The HA molecules seem to have a protective effect that varies inversely with size. That is as the length of the molecule increases, the protective effect declines (FIG. 13b).

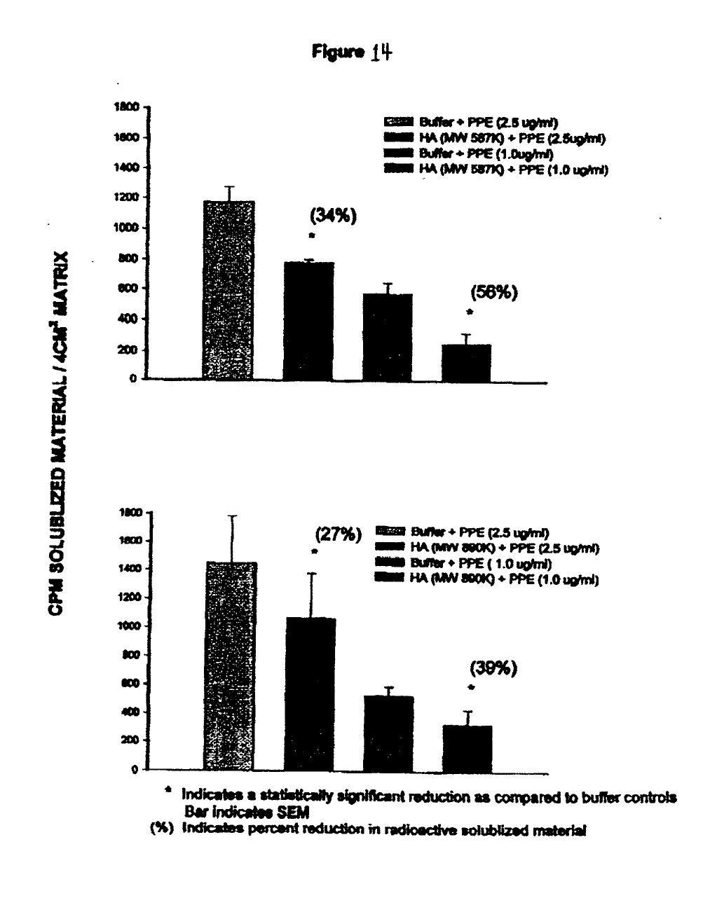

FIGS. 14a and 14b represent the 2 different molecular weight HA specimens tested for their protective effects against two different concentrations of PPE. The concentration of the test solution, the GAG, remains the same (1 mg/ml). FIG. 14a represents the data from digestions of substrate protected with HA 587K and digested with PPE at a concentration of either 1 ug/ml or 2.5 ug/ml. The amount of protection drops as the concentration of PPE is increased, 56% at 1 ug/ml vs. 34% at 2.5 ug/ml. Such effect was seen in earlier testing with HA and is confirmed here. HA 890K demonstrates the same effect starting with a lower protection level as noted above, 39% at 1 ug/ml vs. 27% at 2.5 ug/ml.

FIGS. 14a and 14b also demonstrate a concentration (dose) effect of the enzyme on the Mesogrow-L. As the concentration (dose) of the enzyme is increased there is a commensurate increase in the release of soluble radioactive products from the substrate in both sets of tests. This concentration (dose) response to the enzyme has been demonstrated before and is further confirmed by these tests.

### Example 16

Samples solutions of HA were prepared with varying concentration for a series of different molecular weights. Molecular weights above 200,000 Dalton was measured by intrinsic viscosity and calculated by the Mark-Houwink Equation. Alternatively, molecular weight was measured by HPLC or Light Scattering analysis.

By varying the concentration for a given molecular weight of HA, a range of different viscosities were achieved. These solutions were tested in commercially available nebulizers and the mass median aerodynamic diameter (MMAD) in microns and the geometric standard deviation (GSD) were determined for each tested sample.

Samples solutions of HA were prepared. Concentrations were varied from 0.5 to 2.0 mg/ml at a molecular weight of 890,000, determined by viscometry (Table 2). A range of viscosities from 9.36 to 48.37 centistoke were achieved. These solutions were tested in Whisper, Heart and Misty nebulizers and the mass median aerodynamic diameter (MMAD) in microns and the geometric standard deviation (GSD) were determined for each tested sample. As can be seen from Table 2 below, there was a maximum limit of viscosity above which the HA solution became too viscous to nebulize. This limit is approximately 13-14 cSt for the Whisper nebulizer.

Table 2. Mass Median Aerodynamic Diameter (MMAD) and Geometric Standard Deviation (GSD) for HA Samples of about 890,000 M.W. (L-P9810-1)

| Conc. | Viscosity | | Pressure | MMAD | |
|---|---|---|---|---|---|
| mg/ml | cSt | Nebulizer | psi | (microns) | GSD |
| 2.0 | 48.37 | Whisper | 30 | TVTN* | / |
| 1.0 | 13.94 | Whisper | 30 | TVTN* | / |
| 0.5 | 9.36 | Whisper | 30 | 3.1 | 3.7 |
| 0.5 | 9.36 | Heart | 15 | 5.7 | 4.6 |
| 0.5 | 9.36 | Heart | 30 | 5.7 | 3.8 |
| 0.5 | 9.36 | Misty | 15 | 6.3 | 6.3 |
| 0.5 | 9.36 | Misty | 30 | 4.7 | 4.7 |
| 0.5 | 9.36 | Whisper | 15 | 5 | 5 |
| 0.5 | 9.36 | Whisper | 30 | 2.9 | 3.8 |

*TVTN = too viscous to nebulize

### Example 17

Samples solutions of HA were prepared. Concentrations were varied from 0.5 to 2.0 mg/ml at a molecular weight of 587,000, determined by viscometry (Table 3). A range of viscosities from 7.36 to 32.84 centistoke were achieved. These solutions were tested in Whisper nebulizers and the mass median aerodynamic diameter (MMAD) in microns and the geometric standard deviation (GSD) were determined for each tested sample.

Table 3. Mass Median Aerodynamic Diameter (MMAD) and Geometric Standard Deviation (GSD) for HA Samples of about 587,000 M.W. (L-9411-1)

| Conc. | Viscosity | | Pressure | MMAD | |
|---|---|---|---|---|---|
| mg/ml | (centistoke) | Nebulizer | psi | (microns) | GSD |
| 2.0 | 32.84 | Whisper | 30 | TVTN* | / |
| 1.0 | 13.56 | Whisper | 30 | 4.0 | 4.0 |
| 0.5 | 7.36 | Whisper | 30 | 6.2 | 3.8 |

*TVTN = too viscous to nebulize

### Example 18

Samples solutions of HA were prepared. Concentrations were varied from 0.5 to 2.0 mg/ml at a molecular weight of 375,000 as determined by HPLC (Table 4). A range of viscosities from 3.29 to 12.32 centistoke were achieved. These solutions were tested in Misty nebulizers and the mass median aerodynamic diameter (MMAD) in microns and the geometric standard deviation (GSD) were determined for each tested sample.

Table 4. Mass Median Aerodynamic Diameter (MMAD) and Geometric Standard Deviation (GSD) for HA Samples of about 375,000 M.W. (B-04m81R)

| Conc. | Viscosity | | Pressure | MMAD | |
|---|---|---|---|---|---|
| mg/ml | (centistoke) | Nebulizer | psi | (microns) | GSD |
| 2.0 | 12.32 | Misty | 15 | 5.0 | 5.4 |
| 1.0 | 5.43 | Misty | 15 | 5.2 | 6.1 |
| 0.5 | 3.29 | Misty | 15 | 6.1 | 5.8 |

### Example 19

Samples solutions of HA were prepared. Concentrations were varied from 0.5 to 2.0 mg/ml at a molecular weight of 350,000, determined by viscometry (Table 5). A range of viscosities from 5.56 to 7.14 centistoke were achieved. These solutions were tested in Whisper nebulizers and the mass median aerodynamic diameter (NIMAD) in microns and the geometric standard deviation (GSD) were determined for each tested sample.

Table 5. Mass Median Aerodynamic Diameter (MMAD) and Geometric Standard Deviation (GSD) for HA Samples of about 350,000 M.W. (L-P9706-8)

| Conc. | Viscosity | | Pressure | MMAD | |
|---|---|---|---|---|---|
| mg/ml | (centistoke) | Nebulizer | psi | (microns) | GSD |
| 2.0 | 7.14 | Whisper | 30 | 3.0 | 3.7 |
| 1.0 | 7.09 | Whisper | 30 | 4.0 | 3.6 |
| 0.5 | 5.56 | Whisper | 30 | 3.0 | 3.2 |

### Example 20

Samples solutions of HA were prepared. Concentrations were varied from 0.5 to 5.0 mg/ml at a molecular weight of 220,000, determined by viscometry (Table 6). A range of viscosities from 3.60 to 6.88 centistoke were achieved. These solutions were tested in Whisper and Misty nebulizers and the mass median aerodynamic diameter (MMAD) in microns and the geometric standard deviation (GSD) were determined for each tested sample.

Table 6. Mass Median Aerodynamic Diameter (MMAD) and Geometric Standard Deviation (GSD) for HA Samples of about 220,000 M.W. (L-9711-4)

| Conc. | Viscosity | | Pressure | MMAD | |
|---|---|---|---|---|---|
| mg/ml | (centistoke) | Nebulizer | psi | (microns) | GSD |
| 2.0 | 6.88 | Whisper | 30 | 3.0 | 3.0 |
| 1.0 | 4.01 | Whisper | 30 | 4.9 | 4.5 |
| 0.5 | 3.60 | Whisper | 30 | 4.4 | 4.0 |
| 5.0 | 6.88? | Misty | 15 | 3.37 | 4.8 |
| 2.0 | 6.88 | Misty | 15 | 4.97 | 4.9 |
| 1.0 | 4.01 | Misty | 15 | 4.03 | 4.1 |
| 0.5 | 3.60 | Misty | 15 | 5.23 | 5.0 |

### Example 21

Samples solutions of HA were prepared. Concentrations were varied from 0.5 to 2.0 mg/ml at a molecular weight of 150,000, determined by HPLC and light scattering (Table 7). A range of viscosities from 1.72 to 3.04 centistoke were achieved. These solutions were tested in Whisper nebulizers and the mass median aerodynamic diameter (MMAD) in microns and the geometric standard deviation (GSD) were determined for each tested sample.

Table 7. Mass Median Aerodynamic Diameter (MMAD) and Geometric Standard Deviation (GSD) for HA Samples of about 150,000 M.W. (C-11097)

| Conc. | Viscosity | | Pressure | MMAD | |
|---|---|---|---|---|---|
| mg/ml | (centistoke) | Nebulizer | psi | (microns) | GSD |
| 2.0 | 3.04 | Whisper | 30 | 3.4 | 2.0 |
| 1.0 | 2.24 | Whisper | 30 | 2.1 | 2.3 |
| 0.5 | 1.72 | Whisper | 30 | 2.8 | 2.5 |

### Example 22

Samples solutions of HA were prepared. Concentrations were varied from 1.0 to 5.0 mg/ml at a molecular weight of 140,000, determined by HPLC (Table 8). A range of viscosities from 2.5 to 6.93 centistoke were achieved. These solutions were tested in AeroEclipse, Pari, and Misty nebulizers and the mass median aerodynamic diameter (MMAD) in microns and the geometric standard deviation (GSD) were determined for each tested sample.

Table 8. Mass Median Aerodynamic Diameter (MMAD) and Geometric Standard Deviation (GSD) for HA Samples of about 140,000 M.W. (B-173-EXP001 (A & B))

| Conc. | Viscosity | | Pressure | MMAD | |
|---|---|---|---|---|---|
| mg/ml | (centistoke) | Nebulizer | psi | (microns) | GSD |
| 5.0 | 6.93 | AeroEclipse | 15 | 1.4 | 2.8 |
| 5.0 | 6.93 | AeroEclipse | 30 | 1.3 | 4.8 |
| 2.0 | 3.60 | AeroEclipse | 30 | 3.1 | 3.2 |
| 1.0 | 2.53 | AeroEclipse | 30 | 3.3 | 2.8 |
| 5.0 | 6.9 | Pari | 15 | 2.7 | 3.2 |
| 2.0 | 3.6 | Pari | 15 | 4.3 | 3.4 |
| 1.0 | 2.5 | Pari | 15 | 6.9 | 3.7 |
| 5.0 | 6.9 | Misty | 15 | 4.2 | 3.9 |
| 2.0 | 3.6 | Misty | 15 | 5.2 | 3.4 |
| 1.0 | 2.5 | Misty | 15 | 5.7 | 3.5 |

### Example 23

Samples solutions of HA were prepared. Concentrations were varied from 1.0 to 5.0 mg/ml at a molecular weight of 108,000, determined by light scattering (Table 9). A range of viscosities from 1.9 to 3.7 centistoke were achieved. These solutions were tested in AeroEclipse, Pari, and Misty nebulizers and the mass median aerodynamic diameter (MMAD) in microns and the geometric standard deviation (GSD) were determined for each tested sample.

Table 9. Mass Median Aerodynamic Diameter (MMAD) and Geometric Standard Deviation (GSD) for HA Samples of about 108,000 M.W. (G-9983-1B)

| Conc. | Viscosity | | Pressure | MMAD | |
|---|---|---|---|---|---|
| mg/ml | (centistoke) | Nebulizer | psi | (microns) | GSD |
| 5.0 | 3.7 | AeroEclipse | 15 | 1.9 | 2.4 |
| 5.0 | 3.7 | AeroEclipse | 30 | 2.5 | 2.9 |
| 2.0 | 2.3 | AeroEclipse | 30 | 3.3 | 2.6 |
| 1.0 | 1.9 | AeroEclipse | 30 | 3.7 | 2.3 |
| 5.0 | 3.7 | Pari | 15 | 3.5 | 3.2 |
| 2.0 | 2.3 | Pari | 15 | 6.2 | 3.8 |
| 1.0 | 1.9 | Pari | 15 | 4.2 | 3.4 |
| 5.0 | 3.7 | Misty | 15 | 3.3 | 4.0 |
| 2.0 | 2.3 | Misty | 15 | 6.0 | 3.8 |
| 1.0 | 1.9 | Misty | 15 | 4.6 | 3.7 |

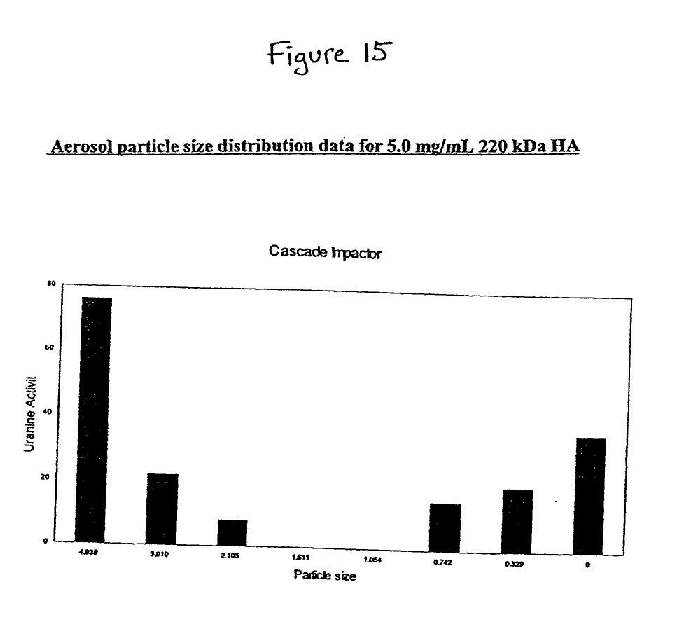

The nebulizer droplet size distributions tended to be bimodal with one mode for sizes larger than about 2 im in aerodynamic diameter and one mode smaller than about 0.5 im (See FIG. 15). Both of these modes are relatively effectively deposited in the lung airways during inhalation and the balance between these modes determines the effective regional deposition of aerosol between the conducting airways and the deep lung These bimodal size distributions are a result of the complex interaction of evaporation phenomena for aerosols from aqueous solutions. Small droplets have higher vapor pressure than larger droplets by virtue of their surface curvature so that small droplets tend to evaporate and larger droplets grow under saturated water vapor conditions. Simultaneously, evaporation is inhibited by the HA in solutions so that the smaller droplets do not completely evaporate and may actually have a higher HA concentration per droplet volume than found in the larger droplets. The result is a bimodal distribution whose exact characteristics depends in part on the selected HA concentration.

Aerosol volumetric output concentration tends to be lower with concentrations of 5 mg/ml than for the lower concentrations (1 mg/ml and 2 mg/ml) all three nebulizers (Misty, Pari, and AeroEclipse). This does not mean that there is proportionately less HA generated at 5 mg/ml since the concentration in solution is much higher. For example, the Misty with 5 mg/ml of HA operated at 15 psig air pressure provides an aerosol of about 15.5 ìl/1 in 5.73 l/min. of air for a total of 15.5 ìl/1×5.73 l/min.=88.8 ìl/min. or 0.0888 ml/min of aerosol generated with the 5 mg/ml concentration. In comparison, at 2 mg/ml HA concentration, the aerosol output was 25.1 ìl/1×5.73 l/min.=144 ìl/min. or 0.144 ml/min. of aerosol. The total HA aerosolized is therefore 0.144 ml/min.×2 mg/ml=0.29 mg/min. of HA aerosol generated with the 2 mg/ml concentration. Although 5 mg/ml is 2.5 times as concentrated as 2 mg/ml, the HA output is only 1.5 more at the higher concentration. If during a twenty minute treatment period, a patient inhales for half of those twenty minutes for the aerosol generated with the 2 mg/ml solution, the inhaled HA would be 0.29 mg/min×10 min.=2.9 mg inhaled. If 60% is deposited in the lung, a total of about 1.7 mg of HA will be deposited in the lungs during this treatment.

The nebulizers acted differently in direct comparison tests. The Misty nebulizer tended to yield undesirable large geometric standard deviations in all tests. The AeroEclipse tended to give smaller droplet size standard deviations, a desirable characteristic.

The use of auxiliary air with the AeroEclipse proved highly successful. The augmentation of aerosol was ideal, with the aerosol concentration remaining about the same with and without auxiliary air. Of course, this means that the aerosol output rate was significantly increased. At a total flow rate of 18 1/min., which is equivalent to the inspiratory demand of a typical person, with 2 mg/ml HA concentration, the aerosol output during inhalation is given by 31.5 ìl/1×18 l/min=567 ìl/min. or 0.576 ml/min. If during a twenty minute treatment period a patient inhales for half of those twenty minutes, the inhaled HA would be 0.575 ml/min.×10 min.×2 mg/ml HA=11.3 mg inhaled. If 60% is deposited in the lung, a total of about 7 mg of HA will be deposited in the lungs during this treatment.

As previously noted, aerosol droplet size distributions with MMAD larger than 10 ìm probably will result in excessive upper respiratory deposition rather than the more desirable alveolar deposition during transoral inhalation by humans. Droplet distributions in the MMAD range from 2 to 4 im are most desirable for therapeutic studies.

Since dilution air is normally required during actual inhalation treatment, some shrinkage of droplets by evaporation may occur, and that can lead to reduced deposition. On the other hand, using a nebulizer that allows auxiliary air to pass through the nebulization zone adding aerosol to that auxiliary air can significantly increase the aerosolization rate and the deposition of HA during a given time period of inhalation treatment. The results found with AeroEclipse nebulizer demonstrate this advantageous use of auxiliary air. That auxiliary air is automatically drawn into the nebulizer from the room in response to the inhalation demand of a patient.

### Example 24

Further, the nebulizer and formulation must be compatible such that the process of producing a respirable aerosol affects no significant changes in HA molecular size or integrity. Examples of such formulation and nebulizer combinations are presented in Table 10.

TABLE 10
Nebulizer and Formulation Compatibility
AeroEclipse nebulizer and formulation compatibility
Nebulizer conditions as described previously for particle size
determinations.
HPLC Conditions: TSK SEC G6000 PW colunm (7.5 × 750 mm)
Mobile phase = 3 mM NaPO4, 0.15M NaCl, pH 7.0,
Run time = 15 min., Injection volume = 100 uL,
Detection = UV at 220 nm;
Flow rate = 1.0 mL/min.

| | Pre-nebulization | Post-nebulization | |
|---|---|---|---|
| Formulation | MW (kD) | MW (kD) | % change |
| Genzyme 9983- | 96,304 | 100,990 | 4.6 |
| P-9708-4A | 387,010 | 393,911 | 1.8 |
| P9711-4 | 215,093 | 207,573 | −3.5 |
| Bayer 173 | 164,729 | 189,062 | 4.6 |

These data show less than +/−5% difference in MW resulting from the aerosolization process, and demonstrate that selection of an appropriate combination of nebulizer and formulation will ensure delivery to the patient of a controlled and specified drug product.

What is claimed is:

1. A method of treating respiratory disorders which comprises administering to a mammal a therapeutically effective amount of a polysaccharide that binds to elastic fibers, thereby preventing enzymes, oxidants, or other injurious agent from contacting and damaging said elastic fibers.

2. The method of claim 1, wherein the polysaccharide is a glycosaminoglycan.

3. The method of claim 2, wherein the glycosaminoglycan is selected from the group consisting of hyaluronic acid, chondroitin sulfate A, chondroitin sulfate B, chondroitin sulfate C, heparan sulfate and heparin.

4. The method of claim 1, wherein the polysaccharide is dextran.

5. The method of claim 1, wherein said administering comprises delivery via a route selected from the group consisting of aerosol inhalation, dry powder inhalation, liquid inhalation and liquid instillation.

6. The method of claim 5, wherein said administering via aerosol inhalation comprises:
preparing a liquid formulation comprising the polysaccharide, wherein the concentration of the polysaccharide is less than about 5 mg/ml and the molecular weight of the polysaccharide is less than about 1.5×10⁶ Daltons;
aerosolizing said liquid formulation to form a breathable mist such that the particle size of the polysaccharide is less than about 10 microns; and
delivering said therapeutically effective amount of the polysaccharide by inhalation of said breathable mist by said mammal.

7. The method of claim 6, wherein the molecular weight of the polysaccharide is less than about 587,000 Daltons.

8. The method of claim 6, wherein the molecular weight of the polysaccharide is less than about 220,000 Daltons.

9. The method of claim 6, wherein the molecular weight of the polysaccharide is less than about 150,000 Daltons.

10. The method of claim 6, wherein said breathable mist is formed by a nebulizer.

11. The method of claim 10, wherein said nebulizer operates at a pressure of at least about 15 psi.

12. The method of claim 10, wherein said nebulizer operates at a pressure of at least about 30 psi.

13. The method of claim 1, wherein the polysaccharide is chemically modified.

14. The method of claim 13, wherein the modification comprises cross-linking.

15. The method of claim 13, wherein the modification comprises addition of sulfate groups.

16. The method of claim 13, wherein the modification comprises addition of carboxyl groups.

17. The method of claim 13, wherein the modification comprises attachment of lipophilic side chains.

18. The method of claim 13, wherein the modification comprises introduction of acetyl groups.

19. The method of claim 13, wherein the modification comprises formation of an ester.

20. The method of claim 13, wherein the modification comprises reaction with a carbodiimide.

21. A method of administering to a mammal a therapeutic formulation comprising a polysaccharide at a selected dose via a respiratory tract, comprising:
formulating a solution comprising the polysaccharide to achieve a controlled polysaccharide size of between about 50,000 and 1.5×10⁶ Daltons at a concentration of less than about 5 mg/ml (w/v) of the polysaccharide;
producing an aerosol of the solution such that a droplet of the aerosol has a median mass distribution size of between about 0.5 to about 10 microns; and
delivering said aerosol into said respiratory tract by inhalation.

22. The method of claim 21, wherein the selected dose of polysaccharide is in a range of about 10 μtg/kg body weight/day to about 1 mg/kg body weight/day.

23. The method of claim 21, wherein the selected dose of polysaccharide is in a range of about 50 μg/kg body weight/day to about 500 μg/kg body weight/day.

24. The method of claim 21, wherein the selected dose of polysaccharide is in a range of about 100 μg/kg body weight/day to about 300 μg/kg body weight/day.

25. The method of claim 21 wherein the solution further comprises a drug.

26. The method of claim 25, wherein the drug is selected from the group consisting of terbutaline, albuterol (salbutamol) sulfate, ephedrine sulfate, ephedrine bitartrate, isoetharine hydrochloride, isoetharine mesylate, isoproteranol hydrochloride, isoproteranol sulfate, metaproteranol sulfate, terbutaline sulfate, procaterol, bitolterol mesylate, atropine methyl nitrate, cromolyn sodium, propranalol, fluroisolide, ibuprofin, gentamycin, tobermycin, pentamidine, penicillin, theophylline, bleomycin, etoposide, captopril, n-acetyl cysteine, verapainil, calcitonin, atriopeptin, alpha.-1 antitrypsin (protease inhibitor), interferon, vasopressin, insulin, interleukin-2, superoxide dismutase, tissue plasminogen activator (TPA), plasma factor 8, epidermal growth factor, tumor necrosis factor, heparin, lung surfactant protein, and lipocortin.

27. The method of claim 21, wherein the polysaccharide is chemically modified.

28. The method of claim 27, wherein the solution further comprises a drug.

29. The method of claim 28, wherein the drug is selected from the group consisting of prostaglandins, amphotericin B, progesterone, isosorbide dinitrate, testosterone, nitroglycerin, estradiol, doxorubicin, beclomethasone and esters thereof, vitamin E, cortisone, dexamethasone and esters thereof, DPPC/DPPG phospholipids, and betamethasone valerete.

30. The method of claim 21, wherein a drug is conjugated to the polysaccharide.

31. A system for delivering a polysaccharide formulation to a respiratory tract of a mammal, comprising:
a mixture comprising a polysaccharide having a molecular weight of between about 50,000 and 1.5×10⁶ Daltons at a concentration of less than about 5.0 mg/ml (w/v) of polysaccharide, and a breathable fluorocarbon propellant;
a cannister adapted to contain said mixture under pressure;
a valve connected to said cannister for regulating delivery of said mixture; and
a nozzle interconnected with said valve for transforming said mixture under pressure into an inhalable aerosol mist when said valve is actuated.

32. The system of claim 31, wherein the polysaccaride in said aerosol mist has a median mass distribution size of between about 0.5 to about 10 microns.

33. The system of claim 31 wherein said mixture further comprises a drug.
